(19) United States
(12) Patent Application Publication
(10) Pub. No.: US 2016/0328993 A1
(43) Pub. Date: Nov. 10, 2016

(54) LOCATION AGNOSTIC PLATFORM FOR MEDICAL CONDITION MONITORING AND PREDICTION AND METHOD OF USE THEREOF

(71) Applicant: HEALTHBITS CORPORATION, Austin, TX (US)

(72) Inventors: Michael Brogioli, Austin, TX (US); Cesar Taylor, Austin, TX (US); Howard Roberts, Austin, TX (US)

(73) Assignee: HEALTHBITS CORPORATION, Austin, TX (US)

(21) Appl. No.: 15/110,643

(22) PCT Filed: Jan. 9, 2015
(86) PCT No.: PCT/US15/10885

§ 371 Date: Jul. 8, 2016

Related U.S. Application Data
(60) Provisional application No. 61/925,516, filed on Jan. 9, 2014.
(60) Provisional application No. 61/925,529, filed on Jan. 9, 2014.
(60) Provisional application No. 61/925,551, filed on Jan. 9, 2014.
(60) Provisional application No. 61/925,555, filed on Jan. 9, 2014.

(51) Int. Cl. G09B 19/00; G08B 21/18; A61M 21/02; A61B 5/08; A61B 5/00

(52) U.S. Cl. CPC G09B 19/003; A61B 5/0816; A61B 5/0022; A61B 5/4824; A61B 5/4839; A61M 21/02; G08B 21/18

(57) ABSTRACT

A system and method of real-time monitoring of medical patient information, both within a medical facility, as well as during in home care. The system and method can include collection of substantial amounts of longitudinal data used to make deductions in trends across a single patients care, care across multiple patients within a facility, and quality of care across given practitioners. The system and method can also include providing information and feedback regarding a patient's perceived quality of care within a facility, as normalized to a given patient's prior experiences. The system and method can also include providing interactive feedback stimuli and patient care experiences to the patient, as well as real time care monitoring systems for practitioners. In embodiments, the present invention can be a system for holistic pain monitoring and prediction, a system for prevention of narcotic diversion, or a magnetometer sensor system for respiratory measurement.

### RELATED PATENT APPLICATIONS

This application claims priority to (a) U.S. Patent Appl. Ser. No. 61/925,516, entitled “Location Agnostic Platform For Medical Condition Monitoring And Prediction And Method Of Use Thereof,” filed on Jan. 9, 2014; (b) U.S. Patent Appl. Ser. No. 61/925,529, entitled “System For Holistic Pain Monitoring And Prediction And Method Of Use Thereof,” filed on Jan. 9, 2014; (c) U.S. Patent Appl. Ser. No. 61/925,551, entitled “System For Prevention Of Narcotic Diversion And Method Of Use Thereof,” filed on Jan. 9, 2014; and (d) U.S. Patent Appl. Ser. No. 61/925,555, entitled “Magnetometer Breathing Sensor System And Method Of Use Thereof,” filed on Jan. 9, 2014. Each of these patent applications is commonly owned by the owner of the present invention.

### FIELD OF INVENTION

The present invention relates generally to a platform for medical conditioning monitoring and prediction. More specifically, the present invention relates to a location agnostic platform for medical conditioning monitoring and prediction and the methods of using this platform. This includes:

In some instances, the present invention relates generally to a system for holistic pain monitoring and prediction and, more specifically, to utilizing a location agnostic platform for holistic pain monitoring and prediction and the methods of using this platform.

In other instances, the present invention relates generally to a system for prevention of narcotic diversion, and more specifically, to monitoring systems and method that monitor and enforce the proper administration of opioid/narcotic medications in hospitals in order to prevent such medications from being used by non-authorized persons.

In further instances, the present invention relates to a magnetometer sensor system for respiratory measurement, and more specifically, relating to systems for monitoring the breathing of sedated patients, especially those receiving narcotic/opioid pain medications that depress or endanger normal breathing.

### BACKGROUND OF INVENTION

“Event processing” (also known as “stream processing”) is a method of tracking and analyzing (processing) streams of information (data) about things that happen (events), and deriving a conclusion from them. Complex event processing (CEP) is event processing that combines data from multiple sources to infer events or patterns that suggest more complicated circumstances. A primary goal of complex event processing is to identify meaningful events (such as opportunities or threats) and respond to them as quickly as possible.

These events may be happening across the various layers of an organization as sales leads, orders or customer service calls. Or, they may be news items, text messages, social media posts, stock market feeds, traffic reports, weather reports, or other kinds of data. An event may also be defined as a “change of state,” when a measurement exceeds a predefined threshold of time, temperature, or other value. Analysts have suggested that CEP will give organizations a new way to analyze patterns in real-time, and help the business side communicate better with IT and service departments.

The vast amount of information available about events is sometimes referred to as the event cloud.

Example of event streams for which an event processing engine or stream processing engine is suited include: (a) business events, such as order instructions received, or credit check succeeded; (b) application events, such as user clicked submit button, or credit check web service indicated success, and (c) system events, such as: order took 1 sec to persist, or credit check service connection pool is low.

“Big data” is the collection of data sets so large and complex that it becomes difficult to analyze and process using on-hand data base tools or traditional data processing tool sets. In addition, the source(s) of data may be varied in quality and resolution, partially complete, and generated in real time from a multitude of sources. Due to the known complexity of such big data systems, care must be taken in system design so as to enforce adherence to consistent design approach, reduce overall and unneeded system complexity, maximize system reuse and increase overall user and system productivity.

Big data architectural components tend to be: model based and architecture driven, facilitating functionality such as data extraction, data ingestion, store, processing, schema, aggregation, messaging, reporting, monitoring, visualization, automation and so forth.

One such solution for processing big data sets was proposed by Google in 2004 called MapReduce. J. Dean et al., “MapReduce: Simplified Data Processing on Large Clusters,” research.google.com/archive/mapreduce-osdi04.pdf (2004). Within MapReduce, queries would be split up and distributed across nodes within the system on which data sets reside. Queries would then be processed in parallel, and results were aggregated during a “reduce” stage. Subsequent to the release of MapReduce, the Apache foundation released an implementation of MapReduce coined “Hadoop.” “1.4 A Brief History of Hadoop,” Hadoop: The Definitive Guide, ed. T. White (publisher O'Reilly 2012).

To a degree, the type and extent of the big data generally varies depending on the capabilities of the organization, application, and infrastructure analyzing the data set itself.

With regard to holistic pain monitoring and prediction, human factors-related iatrogenic harm to patients has been widely recognized in the last decade. In fact, estimates in monetary terms, range from $17 billion to almost $1 trillion as far as costs to society in the United States alone. Respiratory depression secondary to medication administration is one unfortunate example of the above, one that can have devastating consequences including permanent anoxic brain damage and death. Concurrently the healthcare establishment has become more diligent about treating pain.

It is a common mantra that pain level assessment should be treated as another vital sign. However noble this goal, this usually means increased administration of medications capable of depressing the respiratory drive. Patients expect their pain to be controlled adequately when they present to a hospital, but they also expect that it be done safely. Instead, it is estimated that medical errors account for 187,000 deaths per year in the United States, making it the third most common cause of death, behind heart disease and cancer.

Despite hospitals' best efforts, adverse outcomes due to respiratory depression continue. In general, such errors are systems errors, and they have multifactorial etiologies, usually involving some combination of inadequate monitors, insufficient availability of monitors, suboptimal monitoring protocols, and human factors. Having recognized its shortcomings related to safety and reliability, the healthcare industry in primed for new solutions.

Accordingly, there is presently a need for a system for holistic pain monitoring and prediction.

With regard to prevention of narcotic diversion, while opioids and other narcotics are some of the best medications in the treatment of pain, they also have serious and potentially lethal side effects such as respiratory depression, and addiction. It is widely known that opioid/narcotic abuse is a large problem. Narcotic diversion (also referred to as opioid diversion) typically occurs when opioids and other narcotics that have been legally prescribed are used, with or without the patient's knowledge or consent, by a third party. In the outpatient setting, this can occur though theft of illegal sales.

However, narcotic diversion also relates to the diversion of opioids and other narcotics from hospitalized patients to medical providers. Between 2004 and 2008, emergency room visits due to opioids and other narcotics increased from 144,644 to 305,885. Opioid/narcotic overdose is now the second leading cause of unintentional death in the United States, second only to motor vehicle accidents.

This has prompted the Centers for Disease Control and Prevention to consider pharmaceutical opioid/narcotic overdose as a national epidemic. It is also recognized that the incidence of opioid/narcotic abuse is higher among medical providers that among the general population.

The problem when diversion involves medical providers is compounded because the consequences are far-reaching. This is an act of theft, which cheats patients from their pain treatment and directly harms them. The provider abusing the medication may be left impaired while treating other patients in a high-risk environment, leading to a higher chance for medical errors. The addicted provider is personally at risk from the abuse of these medications. Also, the loss of the provider, through disability or death is a great loss to society as there is a shortage of medical providers and it takes years to educate nurses and physicians.

There are several methods currently used to detect diversion in hospitals. However, they are all generally ineffective. These range from self-reporting to detection and reporting of abnormal behavior by co-workers. Additionally, the distribution and dispensing of controlled medications is tightly controlled by pharmacies. When portions of these medications are not used, they are supposed to be wasted and this is supposed to be witnessed. In some institutions unused portions are supposed to be returned to the pharmacy where they may or may not analyze the substance. While these measures may deter diversion, they do not prevent it.

In the case of witnessing wastes, the witness has no way of knowing whether the substance is the opioid/narcotic or not. In other cases, the provider may not report any waste when there was some. In cases where the waste is to be returned to the pharmacies, providers may simply not report or return any waste.

Accordingly, there is presently a need for a system for narcotic diversion monitoring and prediction.

With regard to magnetometer sensor systems for respiratory measurement, the number and frequency of patient operations throughout the world has increased notably over the past 25 years. Today there are approximately 25 million post-operative patients per year in the U.S. alone. Thus, there is an increasing and wide-spread demand for devices, methods, and systems for monitoring and controlling the breathing of postoperative patients while under the influence of powerful narcotics. In addition, sleep apnea is increasingly being diagnosed, and this expanding patient group is more sensitive to narcotics, creating additional risks and demand for timely treatment of endangered breathing.

The Anaesthesia Patient Safety Foundation, the Institute for Safe Medical Practices, the American College of Surgeons and the Joint Commission of Accreditation for Hospitals have determined that patients given narcotics after surgery are being inadequately monitored and are at risk.

Many methods of sensing breathing rates, depths, and profiles are emerging as sensor technology evolves. For instance, U.S. Patent Publ. No. 2002/0097155 to Cassel et al., discloses a combination breathing monitor alarm and audio baby alarm that includes an attachable transmitter forming a main body of a linearly elongated, pliable chest strap of a soft and formable material that is easily wrapable about the chest of an infant. The Cassel device includes a receiver housing receiver control circuitry receiving signals transmitted by the transmitter. U.S. Patent Publ. No. 2011/0290250 to Olson et al., discloses an apparatus and method of monitoring and responding to respiratory depression. The Olson apparatus and method involves a system for monitoring patients, and more specifically post-operative patients receiving narcotics, and an apparatus for automatically delivering a narcotic-reversing agent in response to dangerous respiratory conditions, such as respiratory depression or other undesired consequences caused by reaction to narcotic dosage. U.S. Patent Publ. No. 2011/0295102 to Lakkis et al., discloses a system and method for networked wearable medical sensors.

The current methods of sensing breathing rates, depths, and profiles include visual, acoustic, doppler, and accelerometer techniques, etc. For each of these, there are trade-offs in efficacy, cost, design complexity, limitations, restrictions, power delivery, power consumption, data telemetry, failure modes, and noise floor, noise immunity, electro-magnetic interference to name just a few. For example, a breathing sensor based on an acoustic signature is subject to acoustic interference. Any sensor based on a MEMS accelerometer has a sensitivity threshold due to the quantization error of the A/D that makes it impossible to detect breathing motion when the associated accelerations are below this threshold.

Accordingly, there is presently a need for a system for improved breathing sensor.

### SUMMARY OF INVENTION

The present invention relates to real-time monitoring of medical patient information, both within a medical facility, as well as during in home care. The present invention also relates to the collection of substantial amounts of longitudinal data use to make deductions in trends across a single patients care, care across multiple patients within a facility, and quality of care across given practitioners. The present invention also relates to the patient's perceived quality of care within a facility, as normalized to a given patient's prior experiences. The present invention also relates to providing interactive feedback stimuli and patient care experiences to the patient, as well as real time care monitoring systems for practitioners.

In the context of platform for medical conditioning monitoring and prediction, event streams for which an event processing engine, or stream processing engine, is suited can include a patient's breathing interval has elapsed acceptable threshold (a business event), the medical practitioner has created a new “event” to monitor for a given patient (an application event), and spatially locality amongst undesirable events is occurring between a specific time interval within medical facility (a system event).

Furthermore, for a data collection system within a medical facility the big data can include: real time biometric information, often varied in bit resolution and perhaps missing data in the temporal domain; real time location information sampled from mobile device location services, again possible missing data in the temporal domain; manual/user generated meta-information input to the system with associated time stamps; historical information pertaining to a patient, practitioner, or facility; and RFID information, social networking information, other sources of information.

The present invention is directed to a location agnostic platform for medical conditioning monitoring and prediction, and various methods using said platform. Real-time patient biometric monitoring, in addition to predictive event detection and system analysis may be performed via this platform, via real-time streaming of information back to the platform. Platform supports event detection at the site of the patient, as well as monitoring needs of medical practitioners (computer-to-human communication), and event prediction and detection via monitors (computer-to-computer communication). Said platform shall also support offline data analysis of recorded data streams stored in store in one or more formats.

The platform may generate stimulus feedback directly or indirectly to a patient, as well as receive patient generated information such as quality of pain management and quality-of-service. Platform is also capable of generating, monitoring, and logging of event information within and across medical facilities for consumption by higher layers of application logic.

Platform shall support generation of graphical user interface information, for input and output of user and machine generated information to and from the system. Such graphical user interface devices may be a mix of tablet computers, smart phones, machine to machine devices, all of which shall support custom curation of user views and system monitoring.

Platform devices may be connected via bandwidth brokering engines that delegate computer network bandwidth based on communication priories. Communication priorities may be dependent on criticality of patient condition, number of communication devices within a geolocational region, number of events monitored within a geolocational region, patient group, etc.

Platform shall also support offline data analysis of event metrics and statistics within a medical facility, across regions or user curated groups within a medical facility, and across disparate medical facilities.

In an embodiment of the present invention, the present invention relates to real-time monitoring of medical patient information, both within a medical facility, as well as during in home care. The present invention also relates to the collection of substantial amounts of longitudinal data use to make deductions in trends across a single patients care, care across multiple patients within a facility, and quality of care across given practitioners. The present invention also relates to the patient's perceived quality of care within a facility, as normalized to a given patient's prior experiences. The present invention also relates to providing interactive feedback stimuli and patient care experiences to the patient, as well as real time care monitoring systems for practitioners. This system and method for holistic pain monitoring and prediction of the present invention can utilize the location agnostic platform described herein.

The present invention is able to decrease the role of human factors and provide a mechanism for optimizing protocols and systematizing processes while improving patient engagement and satisfaction. The system of the present invention enforces the current systems and protocols being utilized by hospitals and other caregivers and brings transparency and accountability into play. For instance, when respiratory depression does occur, the present invention implements actions to avoid adverse outcomes and mitigate damage by minimizing time to intervention.

In another embodiment of the present invention, the present invention relates to monitoring systems and method that monitor and enforce the proper administration of opioid/narcotic medications in hospitals in order to prevent such medications from being used by non-authorized persons. This system and method for prevention of narcotic diversion of the present invention can utilize the location agnostic platform described herein.

In another embodiment of the present invention, the present invention relates to a magnetometer sensor system for respiratory measurement that includes a plurality of passive magnets and a plurality of active magnetometers. Persons skilled in the art will understand that, in order to use the sensor in application, the sensor generally must be interfaced to a data acquisition, processing, and control system. This system and method can utilize the location agnostic platform described herein.

In general, in one aspect of the magnetometer sensor system, the present invention can feature a sensor system based on a plurality of magnetic sources attached to a human subject and a plurality of active magnetometer sensors arrayed around the human subject.

In general, in another aspect of the magnetometer sensor system, the present invention features a sensor system that uses passive magnetic sources attached to a human subject requiring no external power source, with no additional passive or active components needed to transmit the signal representative of human breathing.

In general, in another aspect of the magnetometer sensor system, the present invention features a passive magnetic signal source that can be sterilized and re-used in a controlled and sterile environment.

In general, in another aspect of the magnetometer sensor system, the present invention features a method of using such systems and sensors.

In embodiments of the present invention of the magnetometer sensor system, one or more magnets are adhesively attached to the patient, and one or more magnetometers are placed in proximity to the patient and within the field of influence of the magnets. The magnetometers are then interfaced electrically to the data acquisition, processing, and control platform.

The present invention in the aspect of the magnetometer sensor system is especially useful for detection of extremely shallow and low frequency breathing as the magnetometer does not require acceleration to detect movement. The magnetometer makes measurements based on a change in the impinging magnetic field, which can have non-zero values even when the rate of change in the field strength is zero (constant velocity, zero acceleration). Stated alternatively, the magnetometer is sensitive to a change in position of the magnetic and not the acceleration of the magnet. The system reacts only to change in the relative position between the magnet and the magnetometer sensor no matter how slow the movement or how small the acceleration.

The approach of the present invention solves the problem associated with accelerometer based breathing sensors where accumulated error and movement below the noise thresholds of the accelerometer do not accurately detect movements as described. Additionally, the sensor attached to the patient's body is completely passive and requires no power supply, such as batteries, and no wires. Rare earth magnets are suitable for this application because they are inexpensive, small, and can easily be sterilized and packaged in an adhesive container for attachment to the human body.

Furthermore, the present invention may contain a plurality of sensors that enable detection of movement in three dimensional space. The present invention is thus able to leverage magnet and magnetometer technology, paired together, to sense patient chest movement and address many of the engineering, cost, power, sensitivity, and interference issues of alternative methods.

Furthermore, the present invention has life-saving capability even in patients not being treated with narcotics.

The present invention is likewise not limited to a post-operative or any particular type of patient. Indeed, it is contemplated that the sensor may be used on any hospitalized patient and in various home patient situations. While it is contemplated that the financial cost of the sensor is to be relatively low, the risks associated with respiratory depression when compared to any conceivable cost for the device would seem to dictate in favor of widespread and ubiquitous use.

In general, in one aspect, the invention features a system that includes one or more sensors for real-time biometric monitoring of a patient of biometric information. The system further includes one or more input devices for inputting stimulus information of the patient. The system further includes a compute platform in communication with the one or more sensors and the one or more input devices via real-time steaming. The compute platform is operable for performing predictive event detection and analysis utilizing the biometric information and the stimulus information to yield a deduction in real-time. The system further includes an output device in communication with the compute platform operable to provide a real-time stimulus or alert based upon the deduction.

In general, in another aspect, the invention features a method that includes transmitting to a compute platform biometric information of a patient monitored in real time using one or more sensors. The method further includes transmitting to the compute platform stimulus information of the patent using one or more input devices. The method further includes utilizing the compute platform to perform predictive event detection and analysis utilizing the biometric information and the stimulus information to yield deductions in real-time. The method further includes transmitting to an output device a signal to general a real-time stimulus or alert to the patent or a medical practitioner of the patient based upon the deduction. The real-time stimulus or alert prompts an act in response to the deduction.

Implementations of the invention can include one or more of the following features:

The compute platform can be a location agnostic platform for medical conditioning monitoring and prediction.

The real-time stimulus or alert can prompt the patient or medical practitioner to perform an act in response to the deduction.

The real-time stimulus or alert can prompt a device to perform an act in response to the deduction.

The compute platform can include a real-time data and debug logging system.

The system or method can be for holistic pain monitoring.

The system or method can include a respiratory safety module operable for tracking and guiding the patient's condition and medication administration. The system or method can further include an education module operable to provide educational content related to the patient's condition. The system or method can further include an anxiolytic stress reduction module operable to reduce the patient's anxiety and stress level through one or more of education, biofeedback, and medication.

The system or method can link information about medications given to the patient to effects on the patient. The information about medications can be selected from the group consisting of time, dose, type, and combinations thereof.

The compute platform can be operable to analyze and predict about the medication's effect profile across a population of patients.

Information about the medications can be provided to a medical practitioner administering the medication to the patient.

The system or method can include a respiratory tracking module.

The system or method can be for the prevention of narcotic diversion.

The system or method can provide a real-time stimulus or alert of the possibility that a medical provider is diverting a patient's medications for another use.

The system or method is can be for a magnetometer breathing sensor system.

The one or more sensors can include a magnetometer. The system and method can further include one or more magnetic sources secured to the patient.

The one or more magnetic source can include one or more magnets.

The one or more magnetic sources can be within 16 inches of the magnetometer.

The one or more magnetic sources can be within 4 inches of the magnetometer.

The system can be operable for or the method can include assessing when the patient's breathing pattern is inadequate. The system can further be operable for or the method can further include prompting and requesting the patient to breathe. The system can further be operable for or the method can further include providing a stimulus to the patient. The system can further be operable for or the method can further include determining whether further alerts or stimulus need to be deployed. The system can further be operable for or the method can further include determining whether there is a case of unavoidable respiratory arrest. A communication can be provided to a critical response team in response. The stimulus can be an electrical stimulus.

It is also to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates a high level abstraction of the system including an embodiment of the present invention in use, whereby a patient is monitored in real time for various biometric information.

FIG. 2 illustrates more detail of the system illustrated in FIG. 1, which is capable of performing the functionality described for the system.

FIG. 3 illustrates an embodiment of the present invention that is deployed in an environment in which the back end communication resides in a “cloud” compute facility.

FIG. 4 illustrates an example of a bandwidth brokering engine (BBE) module that can be used in embodiments of the present invention.

FIG. 5 illustrates an example of a complex event processing (CEP) engine module (with inputs and outputs) that can be used in embodiments of the present invention.

FIG. 6 illustrates the same system illustrated in FIG. 3 with the patients having varying levels of observation and being given higher or lower priority.

FIG. 7 illustrates a new patient protocol module that can be used in embodiments of the present invention

FIG. 8 illustrates another embodiment of the present invention that is deployed in an environment in which the back end communication resides in a “cloud” compute facility, in which events and data collection services are supported across various facilities within the hospital (other than at the patient's point of care).

FIG. 9 illustrates a further embodiment of the present invention in which the monitoring services are performed across various facilities.

FIG. 10 illustrates a further embodiment of the present invention used to monitor functionality as in-home patient care.

FIG. 11 shows, for an embodiment of the present invention, an example sample message template, as can be represented in an XML format.

FIG. 12 shows, for an embodiment of the present invention, an example of various fields, as can be represented in an XML format.

FIG. 13 shows, for an embodiment of the present invention, an example of a compression techniques that be used on various fields within a given sample message to achieve significant reduction over standard compression techniques.

FIG. 14 illustrates a holistic pain module that can be utilized in an embodiment of the present invention.

FIG. 15 illustrates a respiratory module that can be utilized in the holistic pain module of FIG. 14.

FIG. 16 illustrates a pain medicine administration module that can be utilized in the respiratory module of FIG. 15.

FIG. 17 illustrates a respiratory tracking module that can be utilized in the respiratory module of FIG. 15.

FIG. 18 illustrates a breathing log that can be utilized in the respiratory tracking module of FIG. 17.

FIG. 19 illustrates a vital signs (VSS) tracking module that can be utilized in the respiratory module of FIG. 15.

FIG. 20 illustrates an ID monitor that can be utilized in the pain medicine administration module of FIG. 16.

FIG. 21 illustrates a medical administration protocol that can be utilized in the ID monitor of FIG. 20.

FIG. 22 illustrates an independent double check that can be utilized in the medical administration protocol of FIG. 21.

FIG. 23 is a representation of the type of graphical analysis that could be generated using an embodiment of the present invention. The graph in FIG. 23 shows vital signs represented across time as a change from baseline.

FIG. 24 is a graphical representation of the ability of the system of the present invention to extract details about the pharmacodynamic effects of a medication on a patient.

FIG. 25 illustrates a graphical representation of the ability of the system of the present invention to monitor the effect over time with regards to different dosages.

FIG. 26 is a graphical representation of a standard distribution curve with the spectrum of effects for the dose received that can be created using an embodiment of the present invention.

FIG. 27 is a photograph of a magnetic source that can be used in an embodiment of the present invention.

FIG. 28 is an illustration of another magnetic source that can be used in an embodiment of the present invention.

FIG. 29A is a photograph of the a magnetometer that can be used in an embodiment of the present invention.

FIG. 29B is the internal schematic diagram of the magnetometer of FIG. 29A.

FIG. 30 is a graph that reflects the magnetometer data received using an embodiment of the present invention in which the reading was plotted versus the distance between the magnetic source and the magnetometer.

FIG. 31 illustrates a side view how a magnetic sensor and a magnetometer can be configured in an embodiment of the present invention.

FIG. 32 illustrates a top view of the configuration illustrated in FIG. 31.

FIG. 33 illustrates the configuration shown in FIGS. 31-32 in which the magnetometer is connected to the data acquisition, processing and control platform.

### DETAILED DESCRIPTION

### Location Agnostic Platform for Medical Condition Monitoring and Prediction and Method of Use Thereof

The present invention is directed to a location agnostic platform for medical conditioning monitoring and prediction and the methods of using this platform. While the making and/or using of various embodiments of the present invention are discussed below, it should be appreciated that the present invention provides many applicable inventive concepts that may be embodied in a variety of specific contexts. The specific embodiments discussed herein are merely illustrative of specific ways to make and/or use the invention and are not intended to delimit the scope of the invention.

### Platform

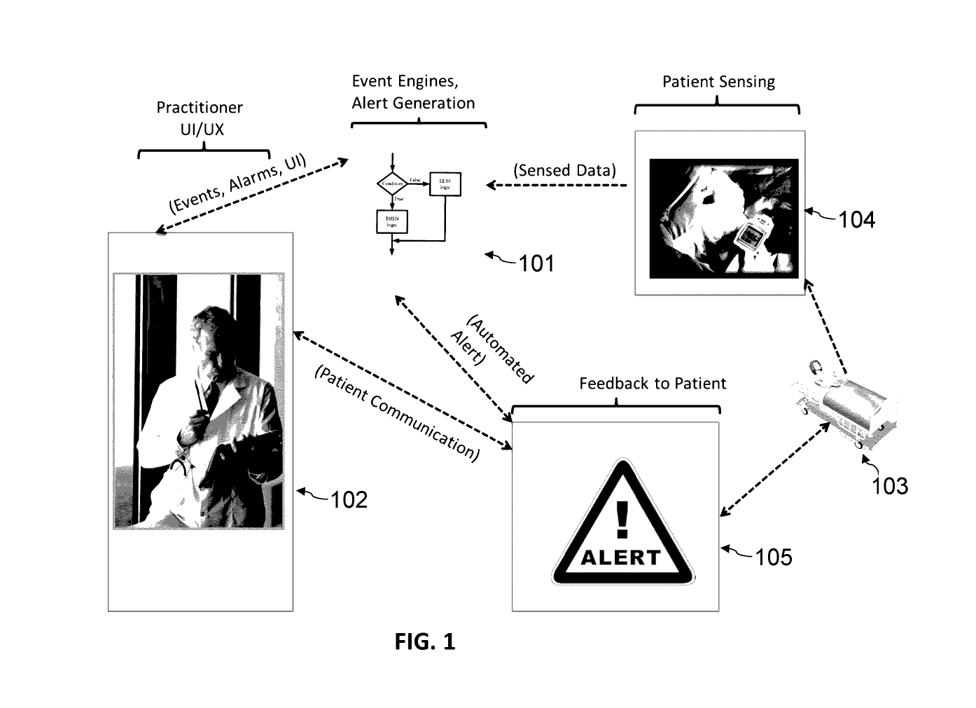

Referring to FIG. 1, this figure illustrates a high level abstraction of the system including an embodiment of the present invention in use, whereby a patient 103 is monitored in real time for various biometric information.

Patient sensing 104 of the patient 103 occurs via various types of biomedical sensors, with associated data communication services providing a stream of patient information to a back end compute/analytics platform 101.

Compute platform 101 is capable of making deductions about information sensed at the site of the patient 103, as well as generating associated views of the data as they pertain the needs of various practitioners (computer to human) and observers (computer to computer).

Moreover, the system includes a patient stimulus component that can receive input either from a practitioner 102, or from back end compute platform 105 to provide information or stimulus feedback to the patient.

In addition, patient 103 may opt to input information to the system via the patient site feedback system 105, such as quality of in facility services, periodic measurements of pain threshold, etc.

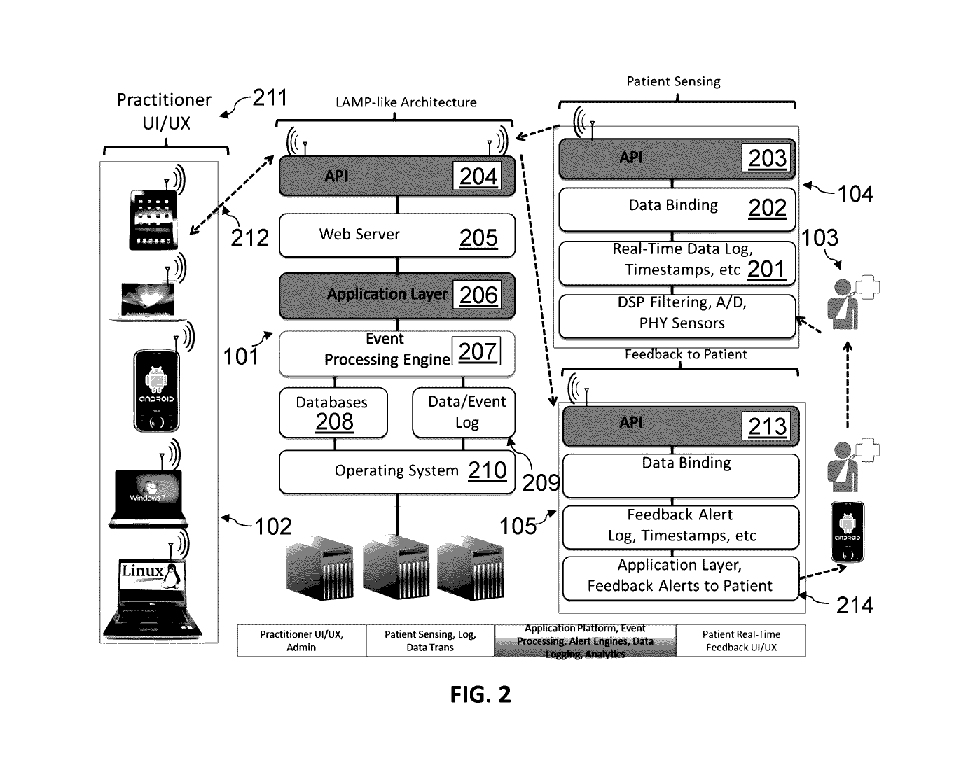

FIG. 2 illustrates more detail of the system illustrated in FIG. 1, which is capable of performing the functionality described for the system.

Patient sensing 104 includes a real-time data and debug logging system 201. Upon sampling of patient data at point of care of the patient 103, various signal processing and formatting occur. The real-time data and debug logging system 201 can provide time stamping of information and system events.

Patient sensing 104 also includes a data binding system 202. Data binding system 202 is used for collecting data binding information events at the patient site (optionally in a structured format) that can be sent in a standard transmission format shared by multiple devices, vendors, and software systems.

Patient sensing 104 also includes an associated API 203 for allowing the query of information and system state as it is maintained at patient site, as well a communication mechanism for transmission of information to the back end compute platform.

Patient sensing 104 also includes DSP filtering, A/D, and PHY sensors.

Compute Platform 101 includes a corresponding API 204, which allows the communication of information into and possibly out of the system.

Compute Platform 101 includes a web server or web services 205 for query of information contained within compute platform 101 by various users from common commodity web browsers, mobile devices. In addition, such services may power machine to machine communication systems.

Compute Platform 101 also includes application logic layer 206 capable of monitoring, recognizing, responding to and logging various events deduced at patient point of care. Additional functionality may include, but is not limited to, time stamped logging of system events and recording of “play back” logs.

Compute Platform 101 also includes event processing engine 207 capable of providing lower layer libraries of event based functionality engines as provided in the application logic layer 206.

Compute Platform 101 includes one or more databases 208 used for application layer logic and various data logging systems 209 for use in the compute platform (including as described above).

Compute Platform 101 also includes a commodity operating system 210 on which application layers and higher layers of logic can operate.

Practitioner 102 has a practitioner device (or devices) 211 (such as a practitioner graphical device) for two way monitoring and custom curating of data views. As shown in FIG. 2, such devices 211 can be a mix of machine-to-machine communication, mobile devices, such as smart phone or tablet compute, and traditional desk top web browsers. Devices 211 can be networked communication 212 from compute platform 101 for observation by practitioner 102.

Feedback system 105 includes a corresponding API 213, which allows the communication of information into and possibly out of the system. The feedback system also includes an application layer 214 such as to provide feedback alert to patient 103. Feedback system 105 also includes features for data binding, feedback alert log, timestamps, etc.

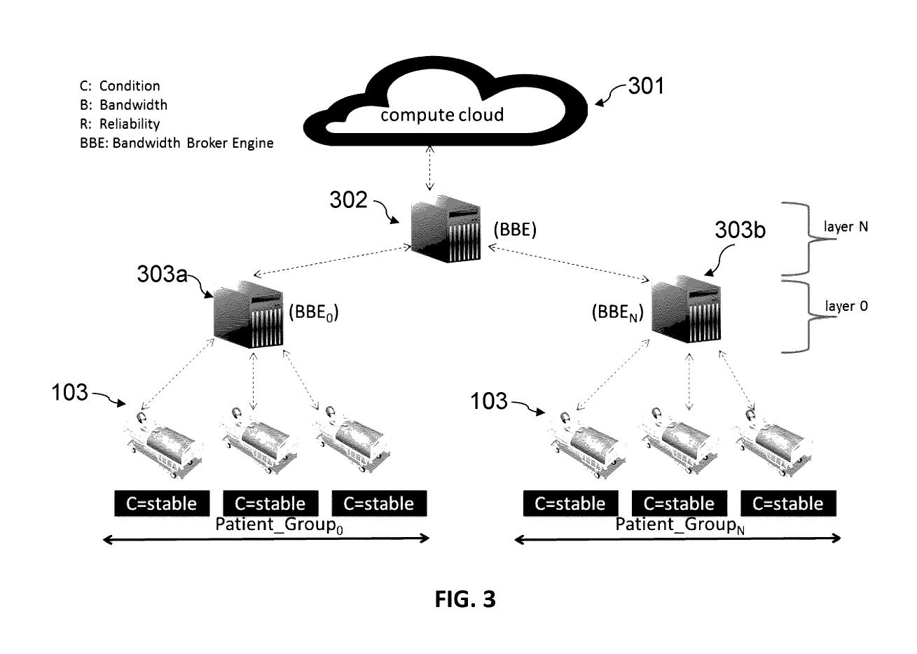

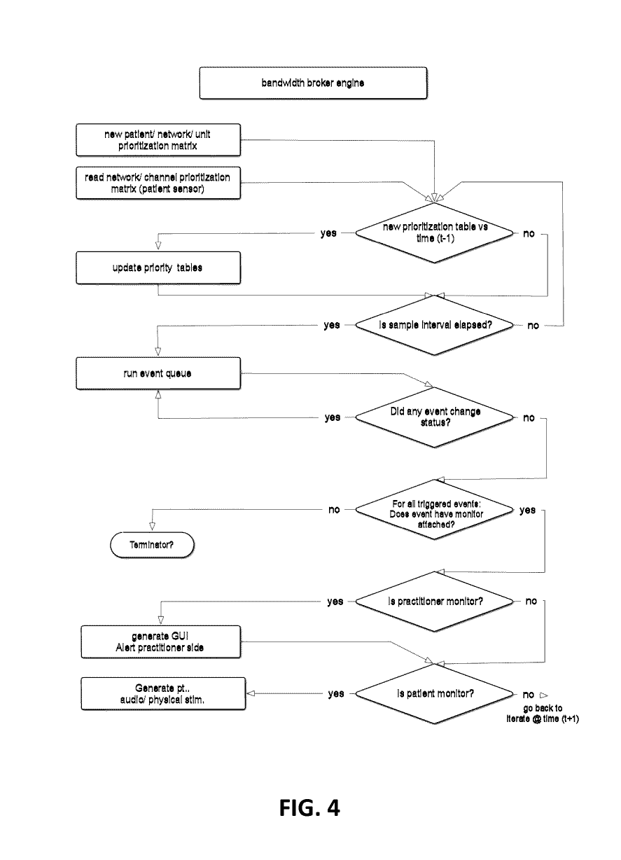

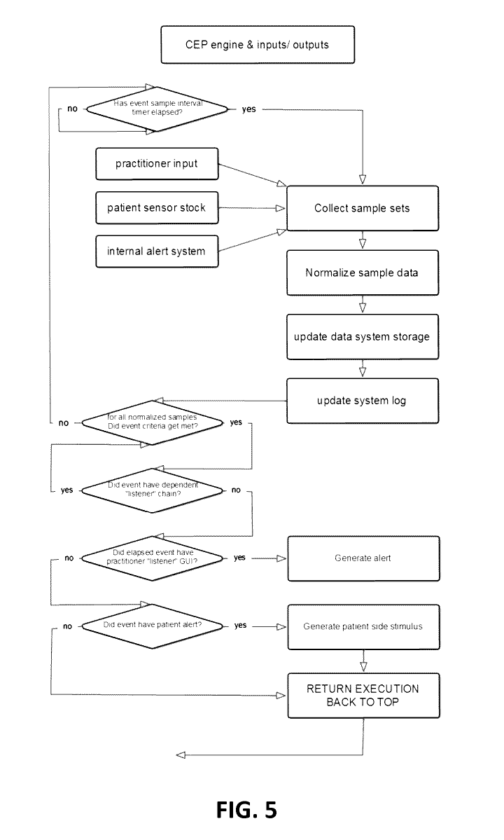

FIG. 3 illustrates an embodiment of the present invention that is deployed in an environment in which the back end communication resides in a “cloud” compute 301 facility. As shown in the embodiment of FIG. 3, various patient 103 points of sensing are connected to a bandwidth brokering engine (BBE) 302 and multiple layers of bandwidth brokering engines (such as BBE 303a and 303b) are tied together to provide dynamically configurable communication to the application server. As shown, the BBE's are associated and have a subscribed number. This is to denote points in the system where bandwidth brokering can be controlled via various means. FIG. 4 illustrates a BBE module that can be used in one or more of the BBEs 302, 303a, and 303b. FIG. 5 illustrates a complex event processing (CEP) engine module (with inputs and outputs) that can be used to combine the data obtained using this embodiment to infer the identify the meaningful events, so that these can be responded to them quickly.

### Medical Facility Platform

As shown in FIG. 3, all patient 103 point of care sensing systems are listed as “stable,” indicating that all care priority levels for patients are equivalent. As such, it would be safe to say in this illustration that the amount of computer network bandwidth dedicated to each patient is equivalent, potentially arbitrated in a “round robin” fashion, etc.

It should be further noted that the bandwidth brokering occurs not only between patients 103 in a patient group, but also across multiple patient groups in this example. This provides a means of grouping patients, or perhaps segregating patients, of a given condition status based on system capability.

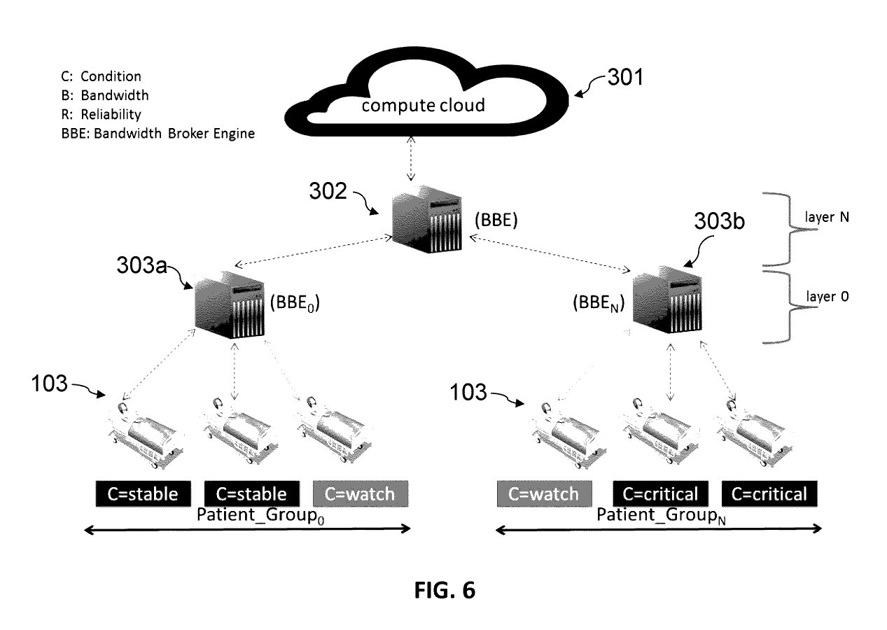

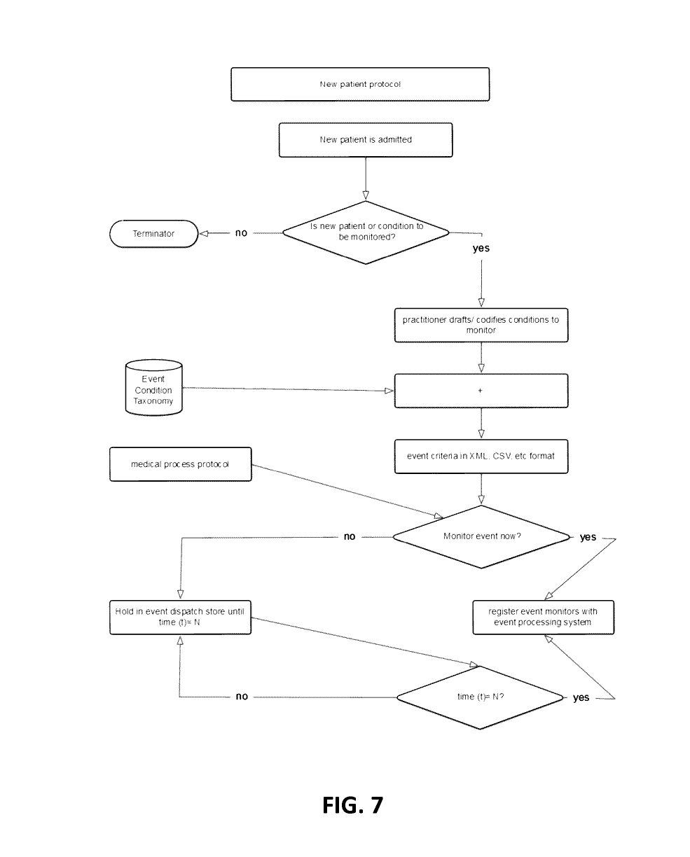

In FIG. 6, a more advanced implementation of the bandwidth brokering topology described for FIG. 3 is detailed. FIG. 6 illustrates the same system illustrated in FIG. 3 with the patients having varying levels of observation and being given higher or lower priority. In this figure, it can be seen that patients 103 of varying levels of observation can be given higher or lower priority, as appropriate. For instance, various patient groups may receive higher or lower priority for network and compute resources, as well as those patients within a given patient group. By way of example, FIG. 7 illustrates a new patient protocol module that can be used when each new patient 103 is admitted.

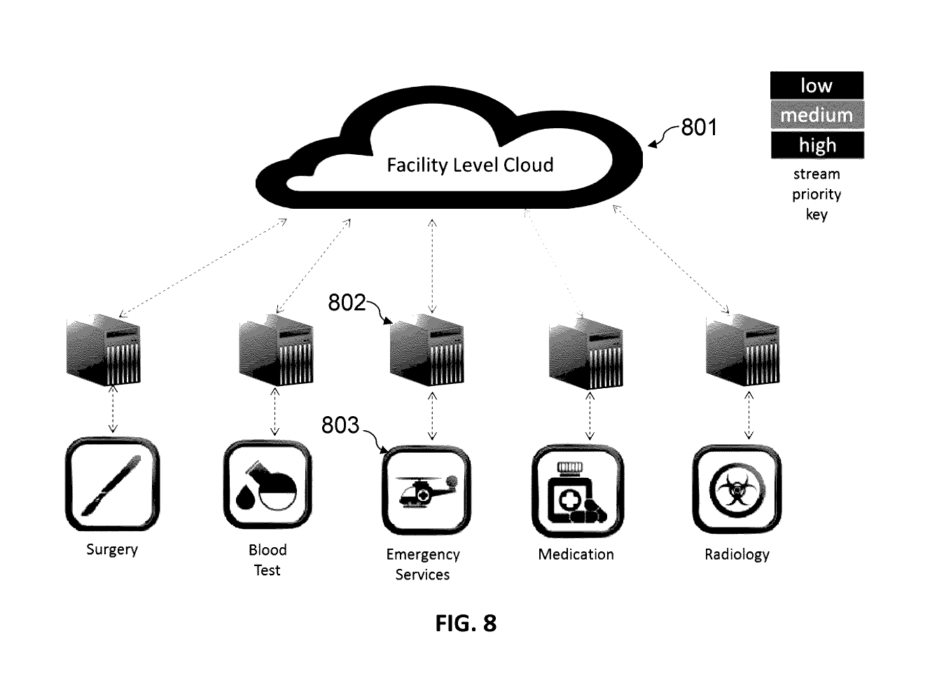

FIG. 8 illustrates another embodiment of the present invention that is deployed in an environment in which the back end communication resides in a “cloud” compute 801 facility, in which events and data collection services are supported across various facilities 803 within the hospital (other than at the patient's point of care). These are connected to a bandwidth brokering engines 802. FIG. 8 expands on the patient centric system topology reflected in FIGS. 3 and 6, whereby in addition to sensing of events at the patient's point of care, similar events and data collection services are supported across various facilities within the hospital. For instance, it may be advantages for the system to monitor quality of care, request response times and other metrics both across a given “unit” within the hospital (space) as well as across time within a given unit (time).

One exemplary use case would be monitoring quality of care across not only individual practitioners and staff members, but also across shifts of employees, etc.

### Multi-Medical Facility Platform

FIG. 6 illustrates a further embodiment of the present invention in which the monitoring services are performed across various facilities. FIG. 6 expands upon the notion of monitoring various events and metrics across units within the hospital both across space and time, and further expands this to monitoring services across various facilities 903 perhaps within a given medical group, or across medical groups. Each of the facilities 903 has a “cloud” 902 that are all themselves in a cloud 901 at the inter-facility level.

### Multi-Home Platform

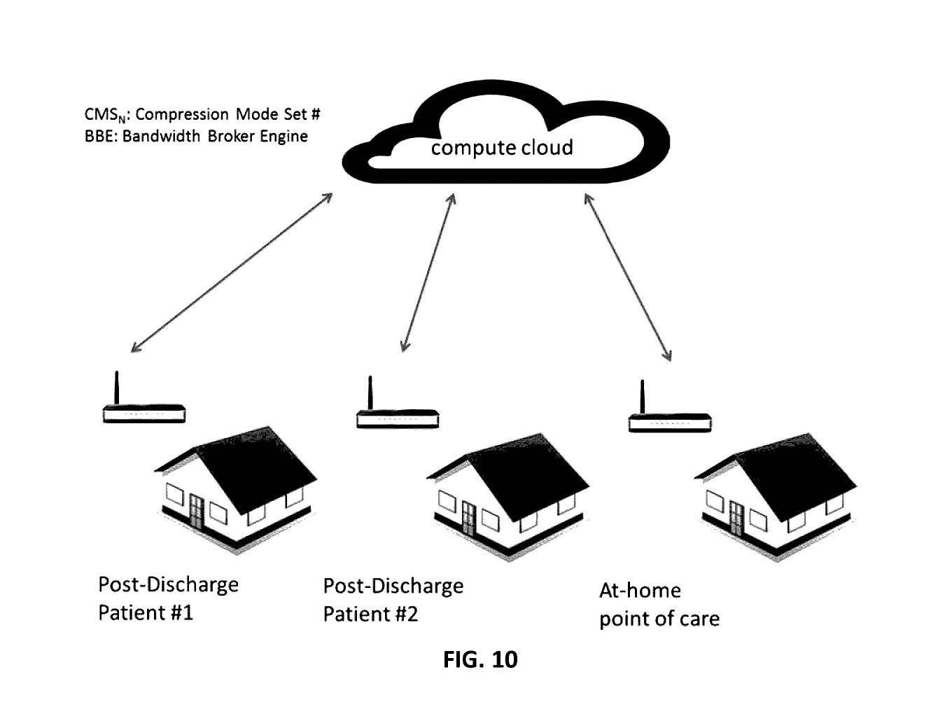

FIG. 10 illustrates a further embodiment of the present invention used to monitor functionality as in-home patient care. In this embodiment, the present invention expands upon the idea of monitoring metrics within a medical facility to similar monitoring functionality as in-home patient care. One exemplary use case would be the reduction of patient readmission rates to hospitals, which may cut into reimbursement rates. For example, consider a patient returning home after in-patient surgery who is required to stand up and walk at a moderate level periodically (such as every 3 hours) while recovering outpatient.

Providing the patient with a mobile compute device, or allowing the patient to use his or her own device with downloadable app, various combinations of accelerometer data, patient input data, practitioner communicated data, and even gamification of the recovery process may be used.

The transmission of medical data over a compute network requires specific timing requirements for accurate detection of events. In addition, since the information being sampled is of a structure typically known a priori, certain compression techniques can be leveraged to reduce the latency and bandwidth required for data transmission. An example of such a use case would be the sampling of heart rate information, which may include a beginning string, a sequence number, a sender identification code, a timestamp, and a sample value. Leveraging a priori information of the sample data, content specific compression techniques may be desirable within the system.

### Messaging and Fields

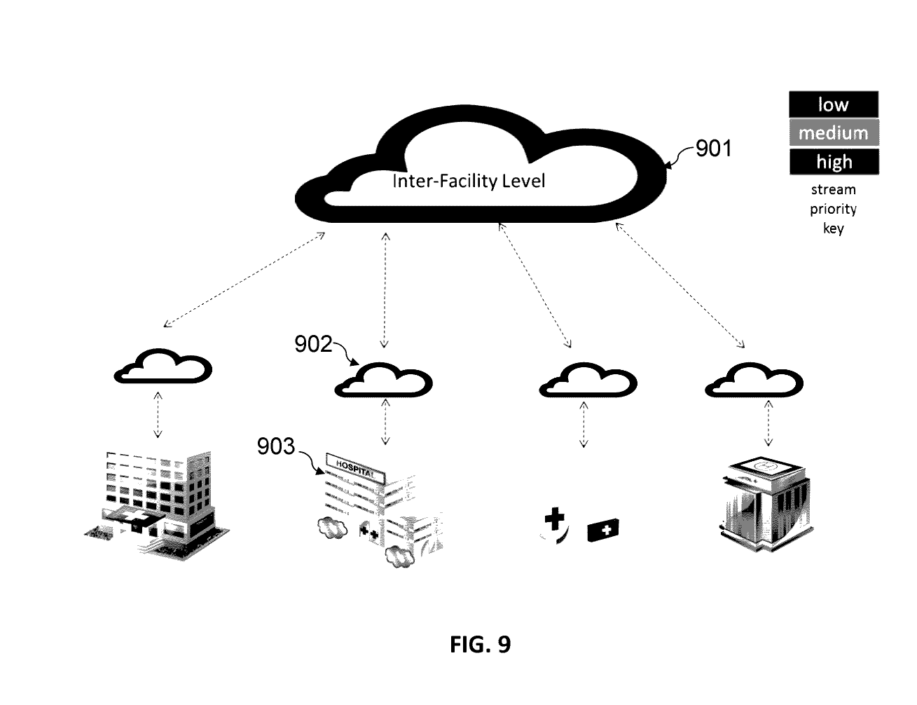

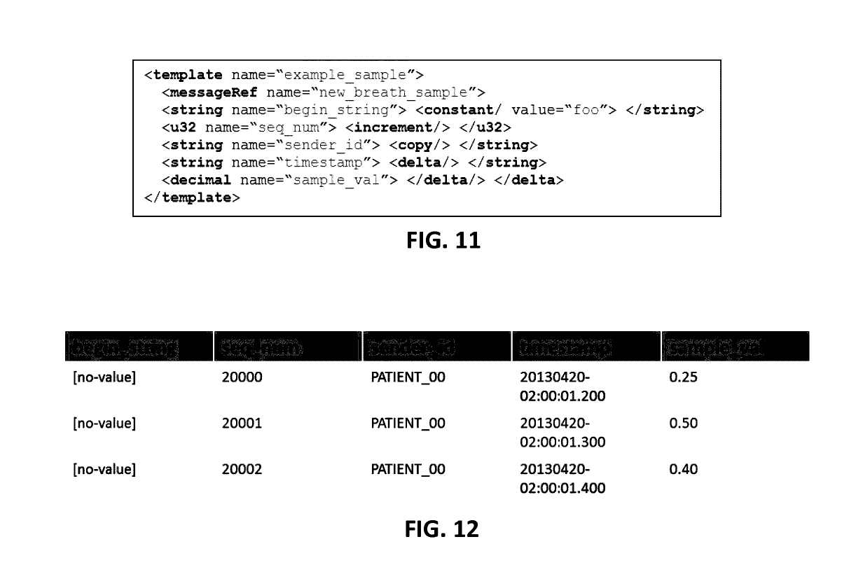

FIGS. 8-9 show, respectively, an example sample message and the various fields, as can be represented in an XML format. For instance, in the example shown in FIG. 11 is an XML formatted template for breath sample taken at the patient site and shown in FIG. 12 is a list of samples taken at patient site, for transmission via the network. These figures detail the XML formatted message, in addition to the various data fields within the message. By knowing the format of the message in advance, various streaming techniques, such as FIX/FAST [Fix Adapted for Streaming—FAST, Protocol Technician Overview, Keith Houstoun, HSBC, FPL Global Technical Committee Co-Chair] can be used. By doing this, significant reductions in the amount of data required per sample packet can be achieved.

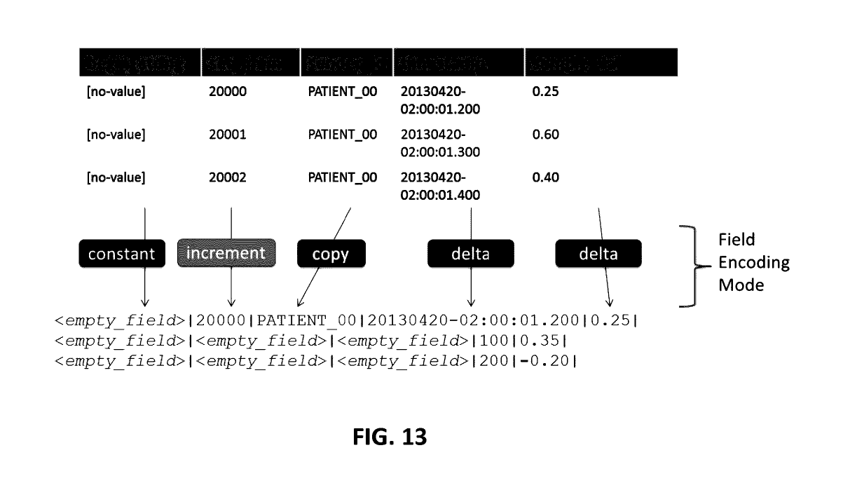

FIG. 13 shows an example of a compression techniques that be used on various fields within a given sample message to achieve significant reduction over standard compression techniques, such as Lempel Ziv coding. FIG. 13 illustrates the various modes used for each field, leveraging the information known beforehand about data attributes of various fields within a message.

System for Holistic Pain Monitoring and Prediction and Method of Use Thereof

In embodiments of the present invention, the present invention is directed to a system for holistic pain monitoring and prediction. While the making and/or using of various embodiments of the present invention are discussed below, it should be appreciated that the present invention provides many applicable inventive concepts that may be embodied in a variety of specific contexts. The specific embodiments discussed herein are merely illustrative of specific ways to make and/or use the invention and are not intended to delimit the scope of the invention.

The present invention is generally utilized with a medical health sensing platform utilizing complex event processing. Thus, in some embodiments, the present invention is a module meant to be part of the application layer logic of a platform, such as the location agnostic platform described above. For representative purposes, embodiments of the present invention in a holistic pain module will be described. Such holistic module can be utilized with the platform described above. However, the systems and methods of the present invention can also be part of another monitoring system or protocol.

### Holistic Pain Module

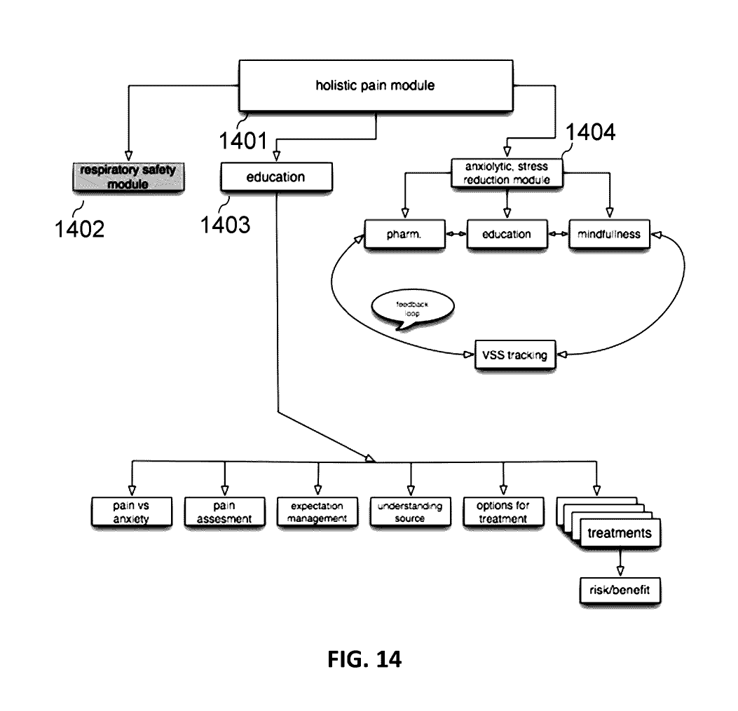

Referring to FIG. 14, this figure illustrates a holistic pain module 1401 that can be used in an embodiment of the present invention and provides an overall view of the approach to pain management in the present invention. Holistic pain module 1401 includes a respiratory safety module 1402, which tracks and guides patients' condition and medication administration. Holistic pain module 1401 also includes an education module 1403, which, through the use of the mobile user interface, provides educational content related to the patient's condition. Holistic pain module 1401 also includes an anxiolytic stress reduction module 1404, which aims to reduce the patient's anxiety and stress level through education, biofeedback and medications.

The overall goal is to provide a comprehensive path for successful and safe management of painful conditions while in hospital. The different components of the assessment and treatment of such conditions are systematized to achieve high reliability. To the extent that anxiety and stress levels contribute to the perception of pain, holistic pain module 1401 aims to diminish such effect.

Moreover, the education component will be able to provide a base of understanding of the painful condition in order to modulate expectations and perception. Patients will become active participants in their care, leading to improved results and satisfaction. Best practices and protocols will be optimized through “big data” analytics and accurate implementation will be facilitated since increased compliance comes from the transparency and accountability built into the system.

### Respiratory Module

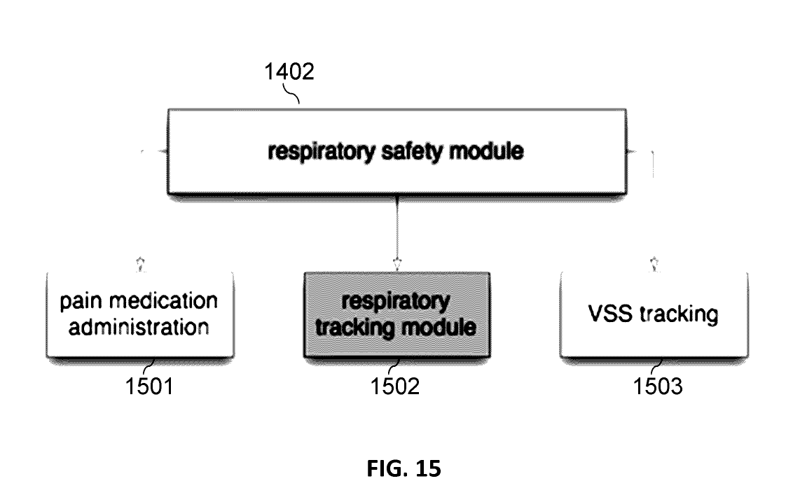

FIG. 15 illustrates a respiratory module 1502 that can be utilized in the holistic pain module 1401 of FIG. 14. The respiratory module 1502 includes a pain medication administration module 1501, respiratory tracking module 1502, and vital signs (VSS) tracking module 1503. The respiratory safety module 1502 oversees the patient's vital signs, pain levels and medication administration. The respiratory tracking module is responsible for monitoring breathing status of patient and taking actions when a fault occurs.

### Pain Medicine Administration Module

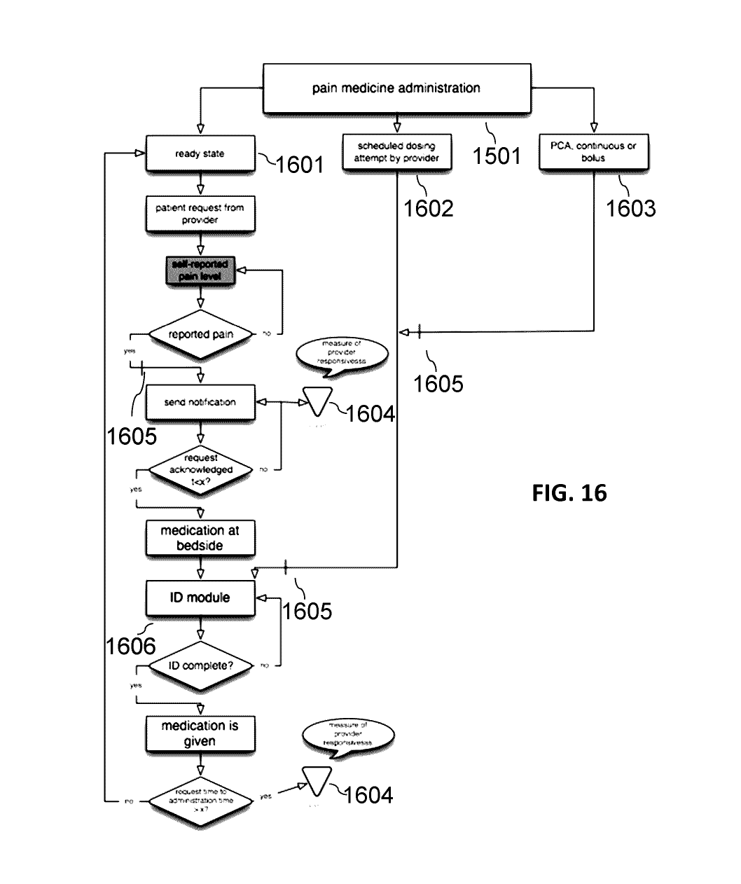

FIG. 16 illustrates a pain medicine administration module 1501 that can be utilized in the respiratory module 1502 of FIG. 15. The pain medicine administration module 1501 describes generally the process by which pain medication is requested and administered. There are generally three instances when a patient would receive pain medication. One instance (instance 1603) is where a patient is already receiving or is connected to an intravenous infusion pump containing a medication such as an opioid. This can be a continuous infusion, a patient-controlled bolus, or a combination of both. The second instance (instance 1602) is where a medical provider, such as a nurse, administers a medication for which there is a physician's scheduled order. The third instance (instance 1601) occurs when a patient experiences either new or increasing pain and the patient requests treatment. The nurse (or other medical provider) at that point will check for pain medications ordered for that patient or if non-existent, he or she will request orders from a physician.

The process of the present invention enforces certain actions from both patient and provider such as prompting for pain levels and requesting identification from provider (such as prompts/requests 1604). When the system detects an unsafe condition as denoted by an alarm originated at this or other modules, the administration of pain medication is stopped in the flowchart of FIG. 16 at stops 1605. Administration of pain medication is allowed only after a series of conditions occur, such as: there are no alerts (such as slow respiratory rate) blocking the process; the patient has reported pain level, and the ID module 1606 has been completed.

Time stamps are typically used to determine response times. This will trigger alerts that can be seen across the organization and that can be used as indicators of patient satisfaction and quality of care. These increased levels of transparency will lead to increased compliance with established protocols as well as improved performance from providers.

### Respiratory Tracking Module

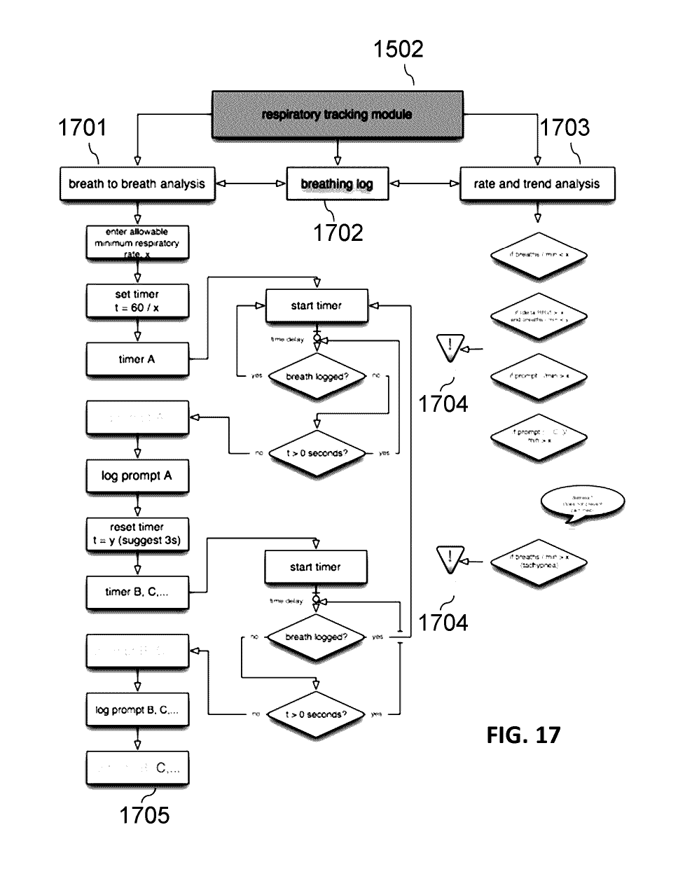

FIG. 17 illustrates a respiratory tracking module 1502 that can be utilized in the respiratory module 1502 of FIG. 15. The respiratory tracking 1502 aims to eliminate the incidence of undetected prolonged respiratory depression. It has three components: breath-to-breath analysis 1701, breathing log 1702 (which captures breath-to-breath events), and analysis 1703 of rates and trends of breath events from the breathing log.

The breath-to-breath analysis 1701 monitors the time interval from one breath to the next. By entering the allowable respiratory rate, a breath-to-breath interval is set and monitored. When the interval is exceeded, an auditory prompt is given to the patient by the system. If there's no compliance within a set interval, the status is elevated and the prompt is repeated with increased urgency. This process repeats with timely compliance re-setting the counter, and lack of compliance incrementally increasing the level of alert and starting a cascade of actions 1705 whereby, when needed (as in the case of prolonged apnea) rescue efforts can be initiated much faster than with current systems.

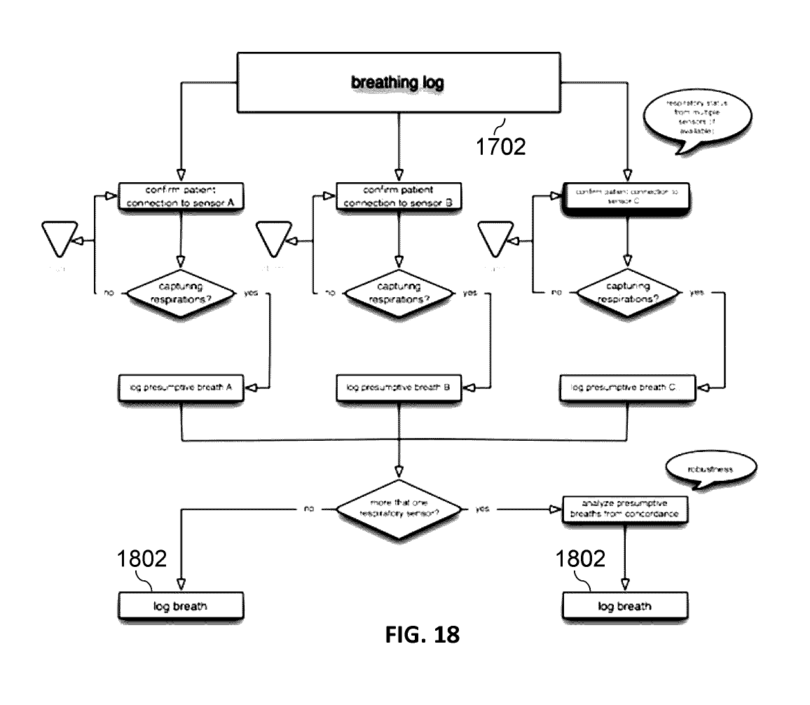

The breathing log 1702, as illustrated in more detail in FIG. 18, records breath events, and in cases where breathing is being sensed from multiple sources, it will run a concordance analysis to provide a more robust determination of whether a breath has occurred or not. Redundancy in sources for capturing breath events is desired for increased robustness of the results and for increased safety in cases where one of more sensors fail. It will be adaptable to varying number of sources.

While apnea mandates a clear response, there are other more subtle clues that can predict impending respiratory depression. The rate and trend analysis 1703 utilizes data from the breathing log 1702 for this purpose. The following are some example scenarios when alerts 1704 typically will be deployed: when respiratory rate falls below a threshold; when the patient has to be prompted by the system multiple times even when compliance occurs; when the rate of escalated prompts exceeds a set number, even when satisfactorily complying; and when the rate of slowing of respiratory rate continues to fall instead of plateauing after administration of medication.

Among other actions, the alerts 1704 will interact at the pain medication administration module 1501 to prevent the patient from receiving further medications that could depress respiration. In instances where the respiratory rate is increased, alerts 1704 will also be deployed as this can indicate a state of distress, either from pain, respiratory, etc. In this case, the system will prompt the patient to check in through user interface device and it will also send alerts 1704 to providers and across a dashboard.

### Vital Signs (VSS) Tracking Module

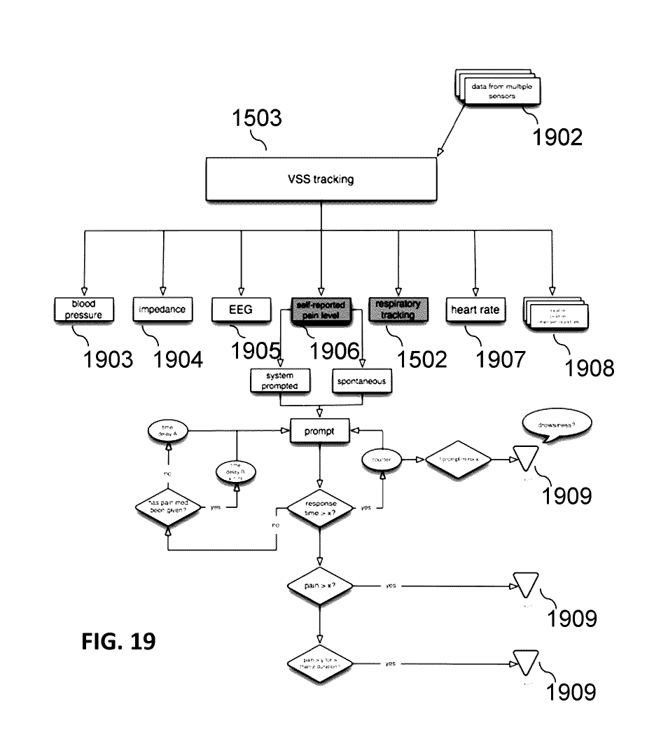

FIG. 19 illustrates a vital signs (VSS) tracking module 1503 that can be utilized in the respiratory module 1502 of FIG. 15. The vitals shown in FIG. 19 (blood pressure 1903, impedance 1904, EEG 1905, self-reported pain level 1906, respiratory tracking 1502, heart rate, 1907, and location, position, and other self-report 1908) are representative only and the actual vital signs recorded will vary according to local availability and needs. The respiratory tracking module 1502 can be included in the VSS tracking module. Pain level assessment also can fall within this module.

Because pain is a subjective perception of discomfort, there is no accurate way to objectively categorize it. The system of the present invention records the pain level as reported by the patient after educating himself or herself about a predetermined convention for reporting pain, such as the visual analog scale (VAS) where pain is reported on a 0-10 scale. Patients can choose (and are encouraged to self-report) their pain level at any time through the mobile user interface. Additionally, the system of the present invention will automatically prompt the patients to provide their pain level at predetermined intervals. Lack of a response for a patient can be analyzed for sedation level. The rate at which the system prompts each of the patients to enter his or her pain level increases once the patient receives pain medication. This information together with other vital signs can then be analyzed to provide insights into the pharmacodynamic effects of individual and combinations of medications across individual patient profiles and across patient populations in order to optimize monitoring protocols. Alerts 609 are also deployed when pain is high for a prolonged length of time.

### ID Module

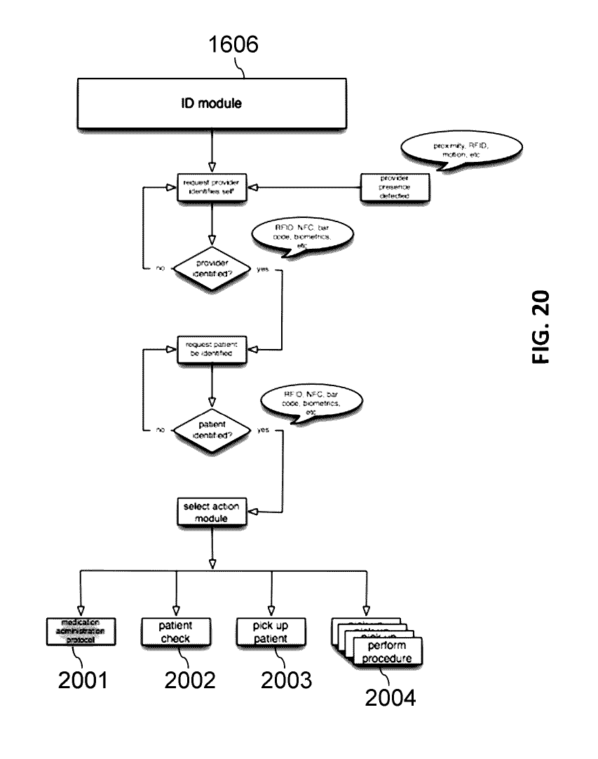

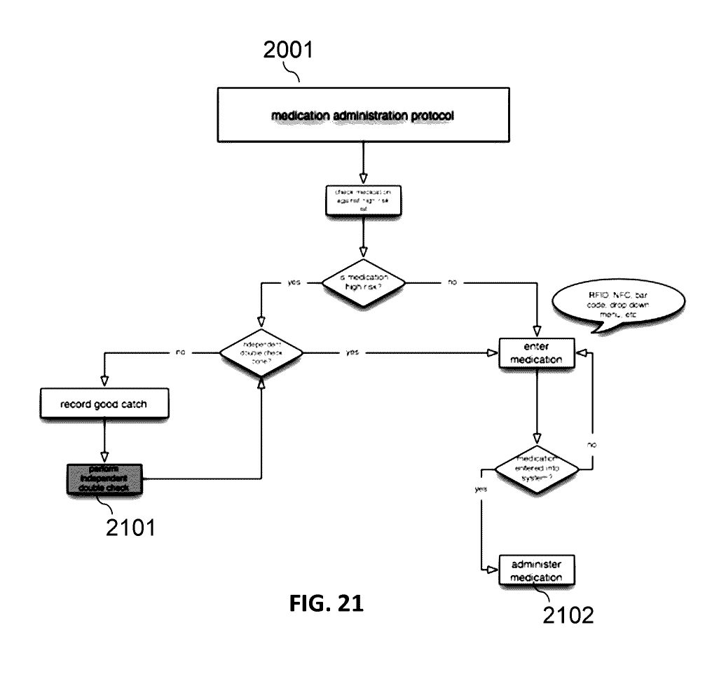

FIG. 20 illustrates an ID monitor 1606 that can be utilized in the pain medicine administration module 1501 of FIG. 16. As shown in FIG. 20, the identity of the provider and the identity of the patient are obtained and logged by the ID module 1606. As shown, patient's encounters are documented by the system prompting for identification of both provider and patient. In cases where a provider's presence in sensed in the room by some mechanism (such as RTLS, proximity sensor, motion sensor, etc.) and when there is temporal correlation with a patient request for assistance or pain medication, the system will automatically prompt for the provider ID. After identification is recorded, the reason for the interaction is entered. In the case of the holistic pain module 1401, one such purpose would be to administer medication, or to assess the patient. In the circumstance when medication will be administered, the medication administration protocol 2001 will be instituted as shown in FIG. 21.

FIG. 21 illustrates a medical administration protocol 2001 that can be utilized in the ID monitor 1606 of FIG. 20.

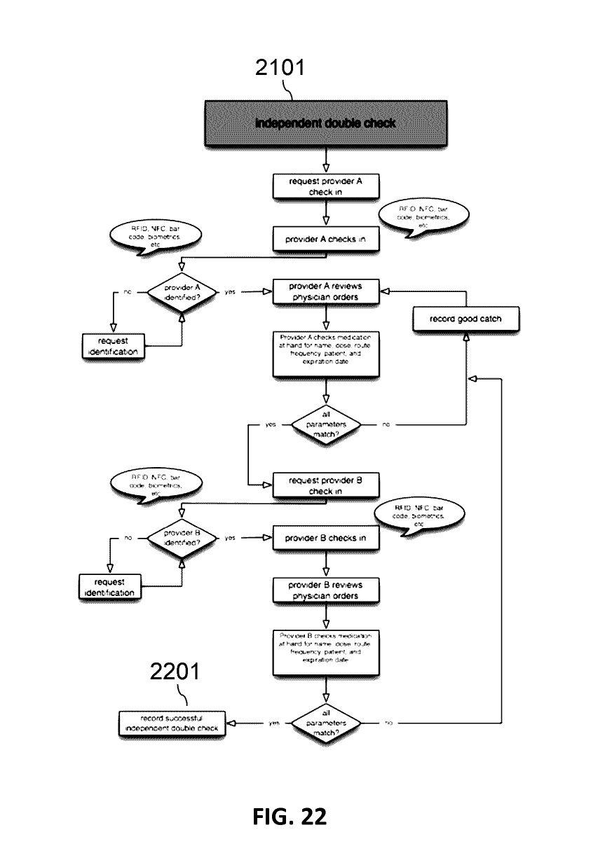

The role of the medication administration protocol 2001 is to determine whether the medication about to be given falls under the high-risk category. If the medication is deemed high-risk, the process will ensure that an independent double check 2101 of the medication has occurred. FIG. 22 illustrates an independent double check that can be utilized in the medical administration protocol of FIG. 21. If the independent double check has not cleared, it will stop the process until that happens. Once cleared, the medication's information will be entered into the system. The method of medication entry will depend on local systems in place: RFID, bar code, etc. where available, drop down menus or manual entry elsewhere. Once entry has been done, the medication will be administered. All events can be recorded and time logged for analysis of provider responsiveness, and compliance with protocols.

Performing an independent double check 2101 is common practice in healthcare institutions when dispensing and administering medications that are considered high risk, such as opioids or insulin. Its purpose is to eliminate human errors where, for example, a patient would be given the wrong medication, wrong dose, or wrong route. In this process, the main provider secures the assistance of a second provider and each (separately and individually) go through the process of checking the medication that has been obtained from the dispensing unit against the written orders, patients information, and allergy list. Some of the problems associated with this procedure are lack of documentation and accountability, as well as unreliable rates of implementation. The systematization of the present invention addresses those issues.

### Representative Analysis/Vial Signs

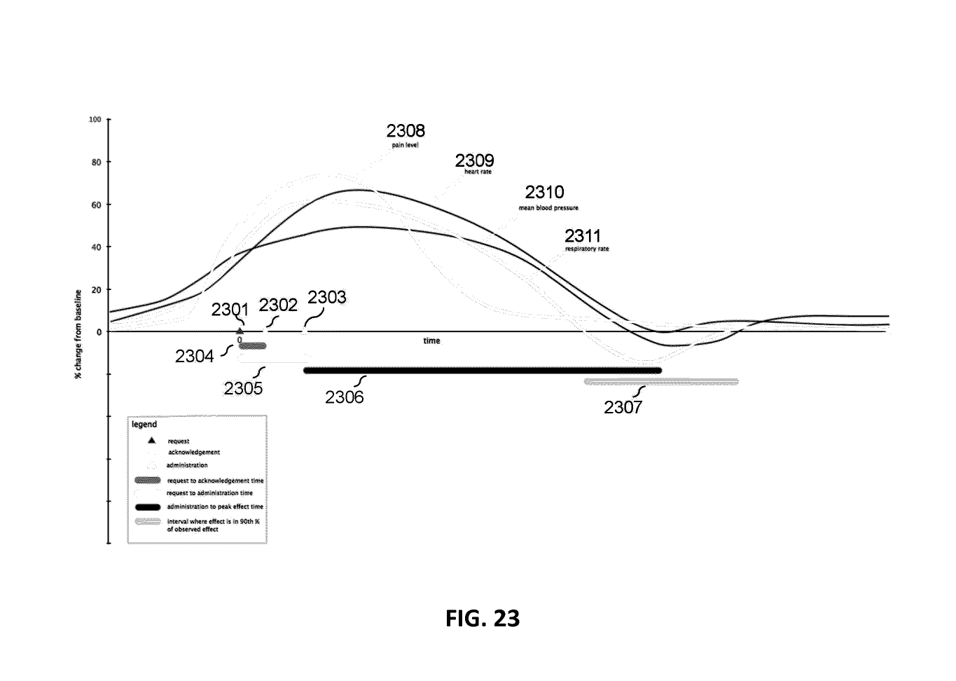

FIG. 23 is a representation of the type of graphical analysis that could be generated using an embodiment of the present invention. The graph in FIG. 23 shows vital signs represented across time as a change from baseline.

Time 2301 is the time at which pain medication was requested. Time 2302 is the time at which the request was received and confirmed by the provider. (Bar 2304 is the time interval it took from the time of the request (at time 2301) and the time of acknowledgement (at time 2302). Time 2303 is the time when the medication was administered. (Bar 2305 is the time interval it took from the time of the request (at time 2301) and the time of administration (at time 2303). Bar 2306 is the time interval it took from administration (at time 2303) to the peak effect time. Bar 2307 is the time interval in which the effect of the administration is in the 90^{th}% of the observed effect.

The effects of the medications administered can be seen and analyzed across the captured vital signs (paint level 2308, heart rate 2309, mean blood pressure 2310, and respiratory rate 2311) for time-to-peak effect as well as duration of peak effect. “Big data” analytics can be used to find trends and patterns across multiple administrations of the same medication, standardized for dose and route of administration as it applies to both general populations as well as specific demographics such as renal insufficiency or opioid tolerant patients.

### Representative Analysis/Drug Effect Over Time

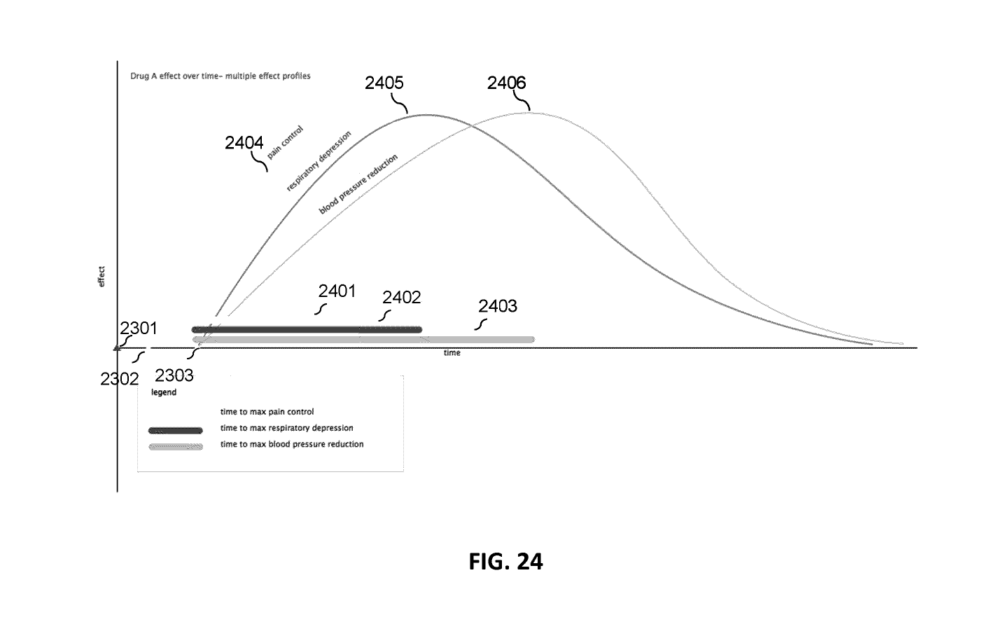

FIG. 24 is a graphical representation of the ability of the system of the present invention to extract details about the pharmacodynamic effects of a medication on a patient. As a general rule, medications seldom have a single effect. In fact, often there are a number of effects other that the one intended. For example, administering an opioid to relieve pain, also have several other effects including those on respiration, heart rate, blood pressure, somnolence, itching, constipation, etc. The ability to determine across populations, the onset-of-effect and peak-effect times for pain control and other undesirable effects will be useful in process improvement efforts.

Similar to FIG. 23, Time 2301, time 2302, and time 2303 are, respectively, the times at which pain medication was requested, at which the request was received and confirmed by the provider, and at which the medication was administered. Bar 2401 is the time interval it took from administration (at time 2303) to maximum pain control. Bar 2402 is the time interval it took from administration (at time 2303) to maximum respiratory depression. Bar 2403 is the time interval it took from administration (at time 2303) to maximum blood pressure reduction. Curve 2404, curve 2405, and curve 2406, are reflectively, the effects of pain control, respiratory depression, and blood pressure reduction over time.

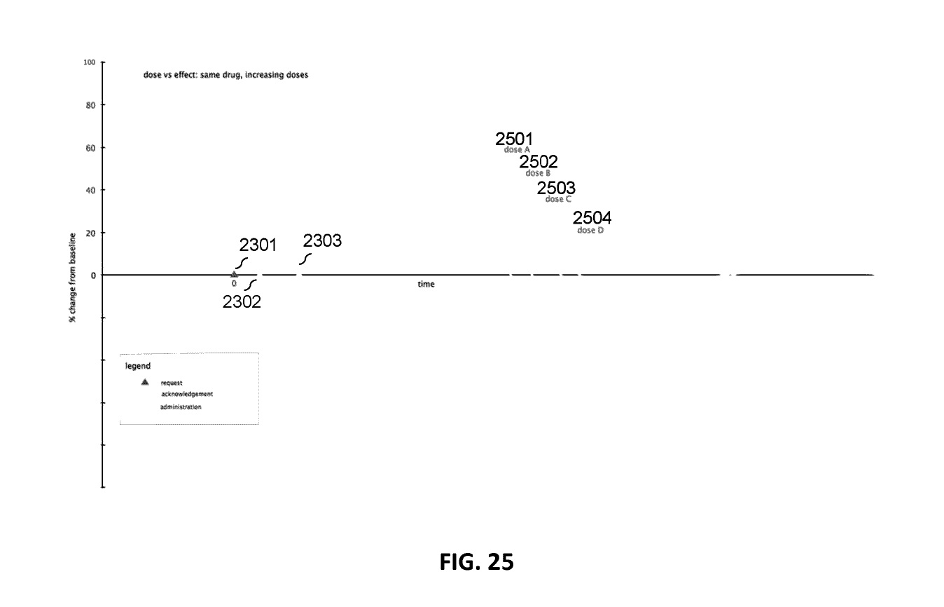

FIG. 25 illustrates a graphical representation of the ability of the system of the present invention to monitor the effect over time with regards to different dosages. Similar to FIG. 23, Time 2301, time 2302, and time 2303 are, respectively, the times at which pain medication was requested, at which the request was received and confirmed by the provider, and at which the medication was administered. Curve 2501, curve 2502, curve 2503, and curve 2504, are the effect of the dose (doses A-D, respectively) as a percent change from a baseline over time.

Currently, patient monitoring protocols fall short due to their one-size-fits-all approach. Typically providers are instructed to check patient status at predetermined intervals regardless of patient demographics, medication, dose or route given. Through analytics, the system of the present invention will produce data-driven suggestions for personalization of monitoring according to patient demographics and medications given according to dose and route. Furthermore, providers can be alerted when medication should be taking effect from a pain relief and from a respiratory depression perspective, thus increasing vigilance during high-risk intervals.

System for Prevention of Narcotic Diversion and Method of Use Thereof

In embodiments of the present invention, the present invention relates generally to a system for prevention of narcotic diversion. The present invention relates to monitoring systems and method that monitor and enforce the proper administration of opioid/narcotic medications in hospitals in order to prevent such medications from being used by non-authorized persons.

While the making and/or using of various embodiments of the present invention are discussed below, it should be appreciated that the present invention provides many applicable inventive concepts that may be embodied in a variety of specific contexts. The specific embodiments discussed herein are merely illustrative of specific ways to make and/or use the invention and are not intended to delimit the scope of the invention.

The present invention is generally utilized with a medical health sensing platform utilizing complex event processing and further utilize with a holistic pain module. Thus, in some embodiments, the present invention is a module meant to be part of the application layer logic of a platform, such as set forth in the Platform patent application, and the holistic pain module, such as set forth in the Pain Monitoring patent application. The present invention involves utilizing such platform and modules for medical sensing utilizing big data analysis and event processing. The application layer logic detects vital signs as well as patients' self-reported pain level. All medication administrations are done so in a controlled manner with the system overseeing and enforcing the process as described.

For representative purposes, embodiments of the present invention in a narcotic diversion prevention module will be described. Such narcotic diversion prevention module can be utilized with the platform set forth in the Platform patent application and utilized with the holistic pain module set forth in the Pain Monitoring patent application. However, the systems and methods of the present invention can also be part of another monitoring system or protocol.

As set forth in the Pain Monitoring patent application, the system and method disclosed therein periodically analyses captured data for trends and patterns correlating opioid and other narcotic dosages in a standardized way (morphine equivalents) to the observed and recorded effects across multiple parameters such as respiratory rate, heart rate, blood pressure, pain level, electroencephalogram, etc. These effects per unit dose of morphine equivalents are also tracked for performance correlation across general and specific patient populations such as healthy, renal failure, liver failure, alcohol abuse, chronic pain, etc. The end result being that through big data analysis, the system can predict the effects a unit dose of medication should have on a given patient. This will be useful in determining safe, effective dosages. Moreover, since that data is also tracked across providers administering the medication, this data analysis provides a novel and efficient way for early detection of narcotic/opioid diversion.

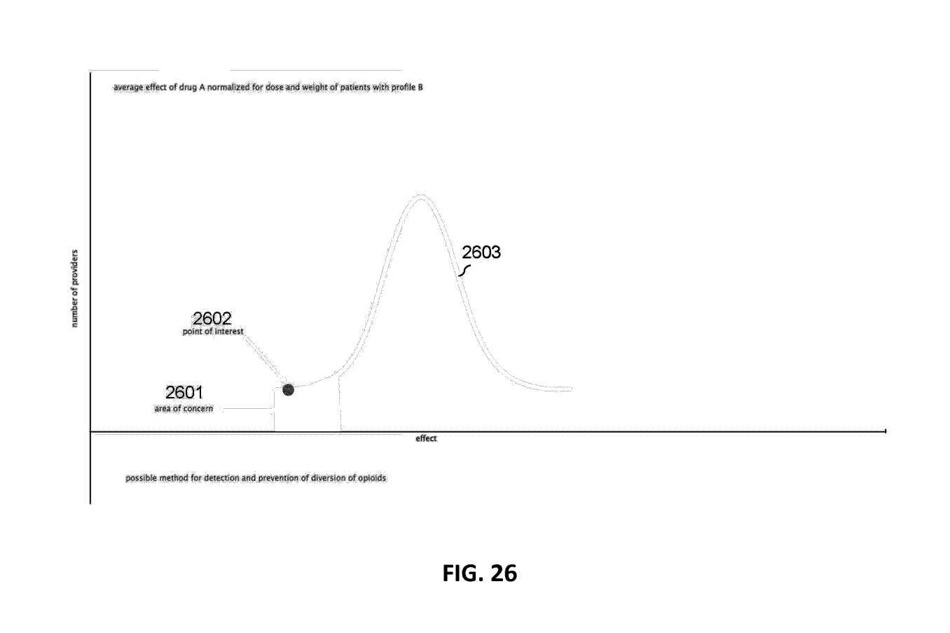

For instance, for a given patient, a standard distribution curve can be created with the spectrum of effects for the dose received. FIG. 26 is a graphical representation of such a standard distribution curve 2603 with the spectrum of effects for the dose received that can be created using an embodiment of the present invention. The area of concern is shown in area 2601, and a point of interest is reflected on distribution curve 2603 at point 2602.

The event processing engine will detect outliers which lie some predetermined amount of standard deviations to the left side of the effect curve, indicating that the medications given by a provider appear to be less effective. This can be done for individual patients and also for all patients treated by that provider. The system can be defined to look for trends as well as set alarms when individual violations occur. For example, a hospital system may decide to allow the platform to notify when a trend with a specific provider is noticed and also when a specific violation of the expected effects occurs with a patient. At that point individual intervention and specific action will be left to the hospital. In addition to serving in opioid diversion detection, the system is also expected to prove itself a deterrent since providers will know that these analytics are occurring in the background, and they'll be less likely to engage in opioid diversion in the first place.

Magnetometer Breathing Sensor System and Method of Use Thereof

In embodiments of the present invention, the present invention is directed to a magnetometer sensor for respiratory measurement and method of using that system. While the making and/or using of various embodiments of the present invention are discussed below, it should be appreciated that the present invention provides many applicable inventive concepts that may be embodied in a variety of specific contexts. The specific embodiments discussed herein are merely illustrative of specific ways to make and/or use the invention and are not intended to delimit the scope of the invention.

The present invention is generally utilized with a medical health sensing platform utilizing complex event processing. Thus, in some embodiments, the present invention is connected to a platform, such as set forth in the Platform patent application.

The system of the present invention involves sensors for monitoring patient breathing. Such a system includes of a plurality of passive magnetic sources and a plurality of active magnetometers.

### Magnetic Source

Generally, the magnet sources require a high field strength to size ratio, a thin, profile, an inert exterior for low corrosion and oxidation rates, the ability to be able to withstand sterilizing temperatures and or liquid environment, and a low cost. An example of a suitable magnet is one that is made of a rare-earth type having high field strength to size ratio, is disk shaped, has a polished exterior, and is relatively low cost (less than a dollar per unit).

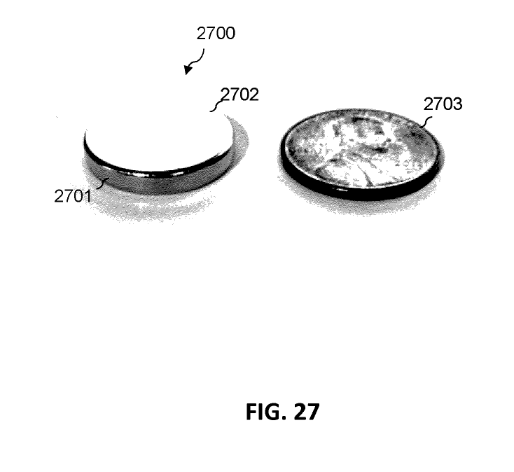

Referring to FIG. 27, FIG. 27 is a photograph of a magnetic source 2700 that can be used in an embodiment of the present invention. The magnetic source is a magnet 2701 having an adhesive pad 2702 that can be used to secure the magnetic source 2700 to the patient. A penny 2703 is also shown in FIG. 27 to provide a frame of reference regarding the relative size of magnet 2701.

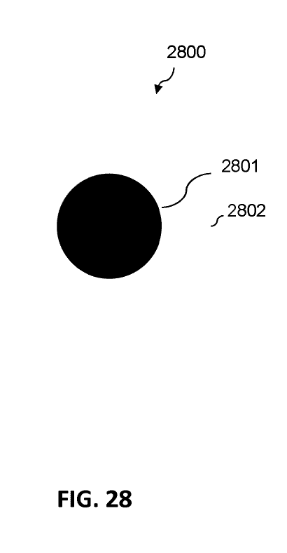

FIG. 28 is an illustration of another magnetic source 2800 that can be used in an embodiment of the present invention. Magnetic source 2800 has a magnet 2801 (which can be the same magnet as shown in FIG. 27 or a different magnet) and a sterile adhesive container 2802 surrounding magnet 2801. Magnet 2801 is placed inside sterile adhesive container 2802 for quick and easy attachment to the patient.

### Magnetometer

Generally, the magnetometers require a small size, a low power, one or more axis of detection (preferably in three dimensions), a low cost, and high sensitivity. An example of a suitable magnetometer is a 3-Axis Digital Compass IC HMC5883L (Honeywell, Plymouth Minn.) (the “Honeywell HMC5883L magnetometer”). The Honeywell HMC5883L magnetometer is a surface-mount, multi-chip module designed for low-field magnetic sensing with a digital interface for applications such as low-cost compassing and magnetometry. The Honeywell HMC5883L magnetometer includes high-resolution HMC118X series magneto-resistive sensors plus an ASIC containing amplification, automatic degaussing strap drivers, offset cancellation, and a 12-bit ADC that enables 1° to 2° compass heading accuracy. The I2C serial bus allows for easy interface. The Honeywell HMC5883L magnetometer is a 3.0×3.0×0.9 mm surface mount 16-pin leadless chip carrier (LCC).

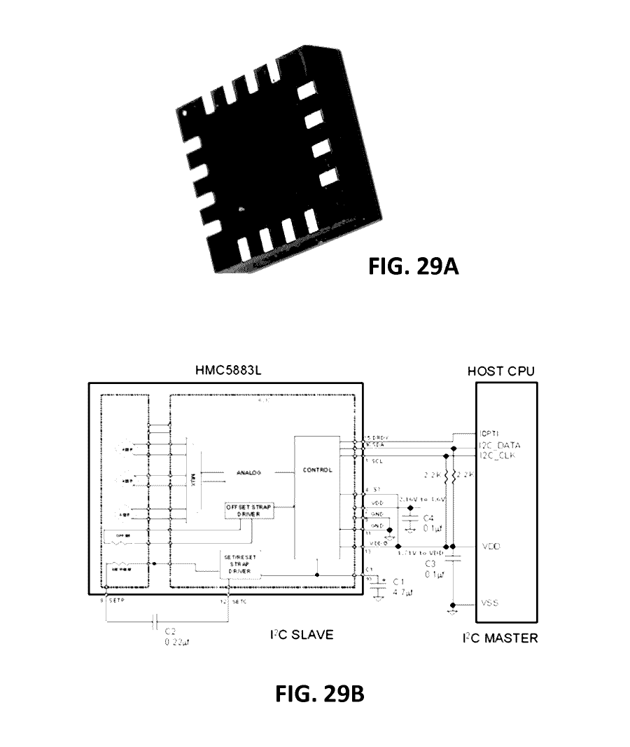

FIG. 29A is a photograph of the Honeywell HMC5883L magnetometer. FIG. 29B is the internal schematic diagram of the Honeywell HMC5883L magnetometer.

### Use of the Magnetometer Breathing Sensor

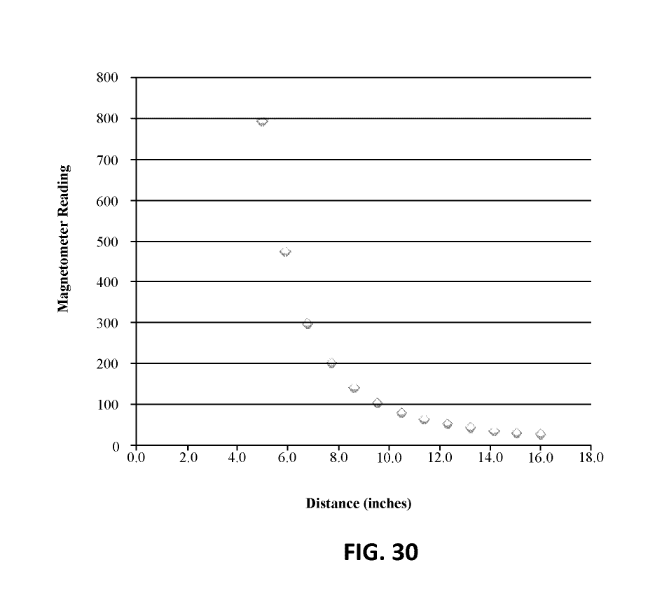

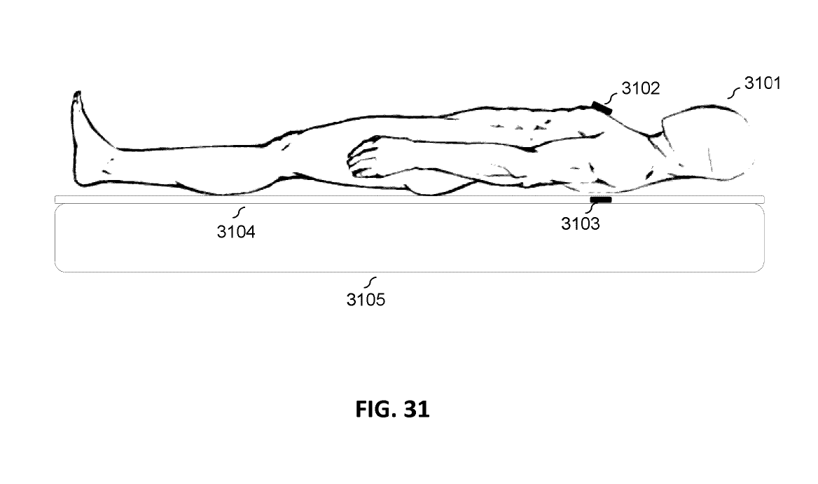

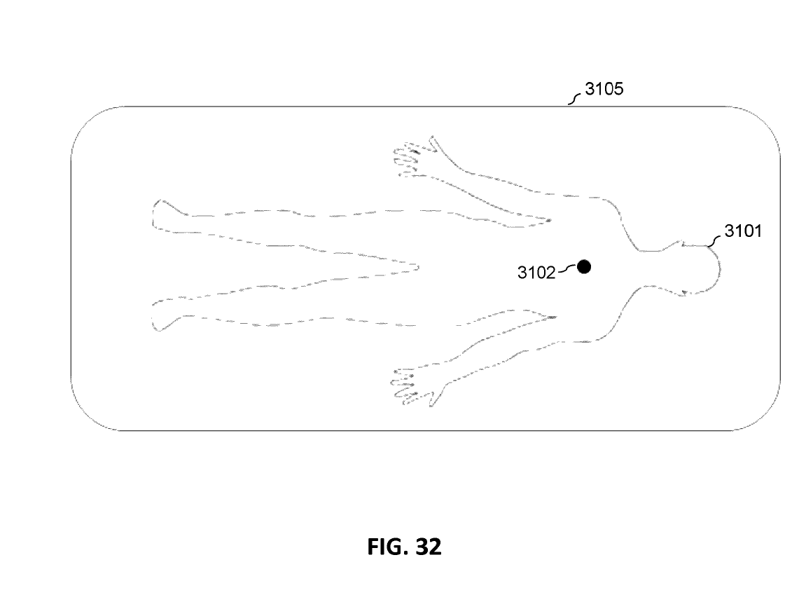

An embodiment of the present was tested using the magnetic source 2700 shown in FIG. 27 and how it moved relative to a Honeywell HMC5883L magnetometer shown in FIG. 29A. Using this embodiment the data in Table 1 was obtained. FIG. 30 is a graph that reflects the magnetometer data of Table 1 in which the average reading was plotted versus the distance between the magnetic source 2700 and the magnetometer. The data of Table 1 was received from testing a male subject having a chest that measured front to back of 10.7 inches. The positioning of the magnetic source 2700 and the magnetometer was similar to that reflected in FIGS. 31 and 32, which are described in more detail below.

TABLE 1

| distance (in) | min | max | avg | resolution |
|---|---|---|---|---|
| 16.0 | 25 | 26 | 25.5 | 0.333 |
| 15.0 | 29 | 30 | 29.5 | 0.250 |
| 14.0 | 34 | 35 | 34.5 | 0.200 |
| 13.0 | 42 | 43 | 42.5 | 0.125 |
| 12.0 | 50 | 51 | 50.5 | 0.125 |
| 11.0 | 61 | 62 | 61.5 | 0.091 |
| 10.0 | 78 | 79 | 78.5 | 0.059 |
| 9.0 | 102 | 103 | 102.5 | 0.042 |
| 8.0 | 140 | 141 | 140.5 | 0.026 |
| 7.0 | 199 | 200 | 199.5 | 0.017 |
| 6.0 | 296 | 297 | 296.5 | 0.010 |
| 5.0 | 474 | 475 | 474.5 | 0.006 |
| 4.0 | 795 | 796 | 795.5 | 0.003 |

This experiment showed the sensitivity of the magnetic source and sensor. The data from the magnetometer was the sum of all three axis. The data indicated that that a distance of 16 inches, the setup can detect movement as small as ⅓ inch or 0.84 cm. It also revealed that it would be possible to position the magnetic source and sensor at a distance of 4 inches and detect movements as small as 3/1000 of an inch or less than 1 tenth of a millimeter. The data exhibited the expected inverse square relationship between magnetic field strength and distance from the source.

The data from Table 1 makes no specific claims with respect to the sensitivity of the magnetometer or the magnetic field strength of the magnet. Accordingly, the results are of a general nature. Both the magnetometer and the magnet are commercially available through common retail sources. The data shows what is possible with little or no calibrating/optimization of the system. The data reflects there will be an upper limit in the separation distance between the magnetometer and the magnetic source (at which point the system becomes ineffective as a breathing sensor). The separation distance should be maintained well below this limit. As a safety feature, the system software can alarm whenever the measured signal falls below the value associated with this upper limit in separation distance.

The data from Table 1 reflect that the magnet and magnetometer pair make an extremely sensitive breathing sensor able to detect an endangered breathing pattern or event. Using the data referenced above, the sensitivity at 11 inches is 0.091 inches or 2.3 mm.

FIGS. 31-32 illustrate a side view and a top view of how a magnetic sensor 3102 and a magnetometer 3103 can be configured in an embodiment of the present invention. The magnetic sensor 3102 is positioned on the chest of the patient 3101. The magnetometer 3103 is positioned below the back of the patient 3101. The magnetometer is position inside a matters lining 3104 that rests on top of bed 3105. The distance between the magnetic sensor 3102 and the magnetometer 3103 could be varied by changing the thickness of the mattress lining 3104.

The embodiment shown in FIGS. 31-32 illustrates a single magnet source and magnetometer pair. Other embodiments of the present invention can have a plurality of magnetic sources and magnetometer sensors arrayed on and around the patient.

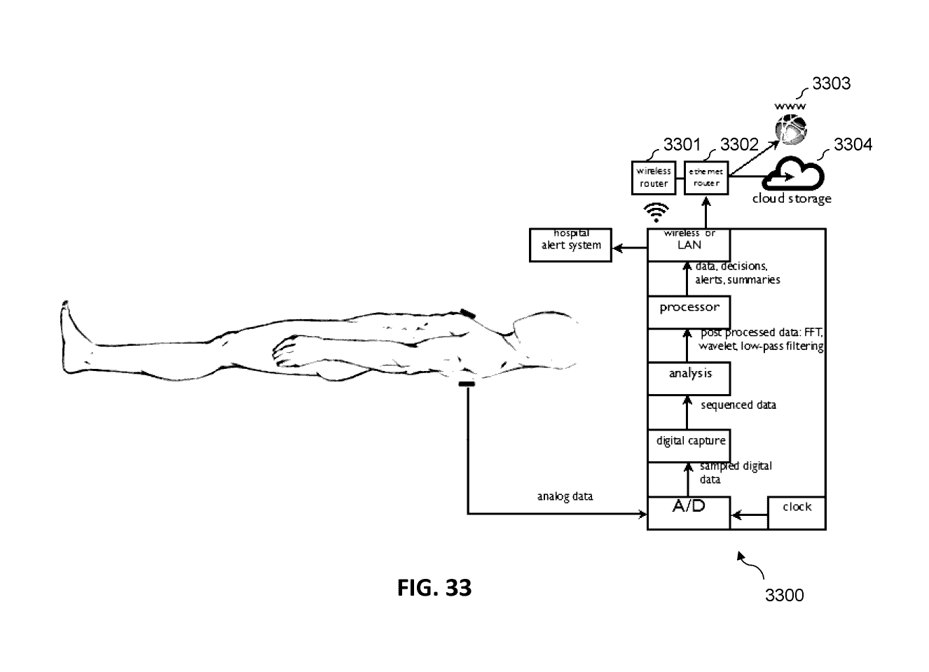

FIG. 33 illustrates the configuration shown in FIGS. 31-32 in which the magnetometer 3103 is connected to the data acquisition, processing and control platform 3300, such as the platform described in the Platform patent application. As show in FIG. 33, the platform 3300 is connect through a router (such as wireless router 3301 or Ethernet router 3302) so that the data can be transmitted over the Internet 3303 or stored in the cloud 3304, as desired.

The examples provided herein and are to more fully illustrate some of the embodiments of the present invention. It should be appreciated by those of skill in the art that the techniques disclosed in the examples which follow represent techniques discovered by the Applicant to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the invention.

The disclosures of all patents, patent applications, and publications cited herein are hereby incorporated herein by reference in their entirety, to the extent that they provide exemplary, procedural, or other details supplementary to those set forth herein. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention.

1. A system comprising:
(a) one or more sensors for real-time biometric monitoring of a patient of biometric information;
(b) one or more input devices for inputting stimulus information of the patient;
(c) a compute platform in communication with the one or more sensors and the one or more input devices via real-time steaming, wherein the compute platform is operable for performing predictive event detection and analysis utilizing the biometric information and the stimulus information to yield a deduction in real-time; and
(d) an output device in communication with the compute platform operable to provide a real-time stimulus or alert based upon the deduction.

2. A method comprising:
(a) transmitting to a compute platform biometric information of a patient monitored in real time using one or more sensors;
(b) transmitting to the compute platform stimulus information of the patent using one or more input devices;
(c) utilizing the compute platform to perform predictive event detection and analysis utilizing the biometric information and the stimulus information to yield deductions in real-time; and
(d) transmitting to an output device a signal to general a real-time stimulus or alert to the patent or a medical practitioner of the patient based upon the deduction, wherein the real-time stimulus or alert prompts an act in response to the deduction.

3. The system of claim 1, wherein the compute platform is a location agnostic platform for medical conditioning monitoring and prediction.

4. The system of claim 1, wherein the real-time stimulus or alert prompts the patient or medical practitioner to perform an act in response to the deduction.

5. The system of claim 1, wherein the real-time stimulus or alert prompts a device to perform an act in response to the deduction.

6. The system of claim 1, wherein the compute platform comprises a real-time data and debug logging system.

7. The system of claim 1, wherein the system or method is for holistic pain monitoring.

8. The system of claim 7, wherein the system or method comprises:
(a) a respiratory safety module operable for tracking and guiding the patient's condition and medication administration;
(b) an education module operable to provide educational content related to the patient's condition; and
(c) an anxiolytic stress reduction module operable to reduce the patient's anxiety and stress level through one or more of education, biofeedback, and medication.

9. The system of claim 7, wherein the system or method links information about medications given to the patient to effects on the patient.

10. The system of claim 9, wherein the information about medications is selected from the group consisting of time, dose, type, and combinations thereof.

11. The system of claim 9, wherein the compute platform is operable to analyze and predict about the medication's effect profile across a population of patients.

12. The system of claim 9, wherein information about the medications is provided to a medical practitioner administering the medication to the patient.

13. The system of claim 1, wherein the system or method comprises a respiratory tracking module.

14. The system of claim 1, wherein the system or method is for the prevention of narcotic diversion.

15. The system of claim 14, wherein the system or method provides a real-time stimulus or alert of the possibility that a medical provider is diverting a patient's medications for another use.

16. The system of claim 1, wherein the system or method is for a magnetometer breathing sensor system.

17. The system of claim 16, wherein the one or more sensors comprise a magnetometer and the system and method further comprises one or more magnetic sources secured to the patient.

18. The system of claim 17, wherein the one or more magnetic source comprise one or more magnets.

19. The system of claim 17, wherein the one or more magnetic sources are within 16 inches of the magnetometer.

20. The system of claim 17, wherein the one or more magnetic sources are within 4 inches of the magnetometer.

21. The system of claim 17, wherein the system is operable for or the method comprises:
(a) assessing when the patient's breathing pattern is inadequate;
(b) prompting and requesting the patient to breathe;
(c) providing a stimulus to the patient;
(d) determining whether further alerts or stimulus need to be deployed; and
(e) determining whether there is a case of unavoidable respiratory arrest, wherein a communication is provided to a critical response team in response.

22. The system of claim 21, wherein the stimulus is an electrical stimulus.

23. The system of claim 1, wherein the system is operable for or method comprises:
(a) assessing when the patient's breathing pattern is inadequate;
(b) prompting and requesting the patient to breathe;
(c) providing a stimulus to the patient;
(d) determining whether further alerts or stimulus need to be deployed; and
(e) determining whether there is a case of unavoidable respiratory arrest, wherein a communication is provided to a critical response team in response.
